(19) United States
(12) Patent Application Publication
(10) Pub. No.: US 2022/0165407 A1
(43) Pub. Date: May 26, 2022

(54) CUSTOMIZABLE COMMUNICATION PLATFORM WITH ALERT TAG PRIORITIZATION AND REVIEW

(71) Applicant: CAREMINDR Corporation, Los Gatos, CA (US)

(72) Inventors: Harry Raymond Soza, San Jose, CA (US); Irina Yermilov, Los Angeles, CA (US)

(21) Appl. No.: 17/100,690

(22) Filed: Nov. 20, 2020

(51) Int. Cl. G16H 40/67; G16H 50/20; G16H 10/60

(52) U.S. Cl. CPC G16H 40/67; G16H 10/60; G16H 50/20

(57) ABSTRACT

One example method includes linking a user equipment and a health care provider server, requesting via a cloud server a patient response to a query of a health related issue sent to a user equipment of a patient, receiving via the cloud server a patient response to the query from the user equipment, determining via the cloud server a review criticality of the patient response based on the health related issue, flagging via the cloud server the patient response as a critical flagged response if the review criticality exceeds a predetermined criticality threshold for the health related issue, disclosing via the cloud server to a local health care provider server the critical flagged response in an order of criticality and outreaching via the cloud server to the local health care provider server and a local health care provider device the patient response if the review criticality indicates substantial public hazard.

### BACKGROUND

Conventionally, an approach to providing users with ongoing communications regarding a plan or other courses of action may leave the majority of the work to the user. A lack of action taken by a professional services provider or a user may place the user at additional risk.

### SUMMARY

Example embodiments of the present application provide at least a first example method of the present application includes at least one of requesting via a cloud server at least one patient response to at least one query of at least one health related issue sent to a user equipment of a patient, receiving via the cloud server at least one patient response to the at least one query from the user equipment, determining via the cloud server a review criticality of the at least one patient response based on the at least one health related issue, flagging via the cloud server the at least one patient response as at least one critical flagged response if the review criticality exceeds a predetermined criticality threshold for the at least one health related issue, disclosing via the cloud server to a local health care provider server the at least one critical flagged response in an order of criticality thereby increasing a workflow efficiency and outreaching via the cloud server to at least one of the local health care provider server and a local health care provider device the at least one patient response if the review criticality indicates a substantial public hazard.

A second example embodiment of the present application provide at least a non-transitory computer readable medium comprising instructions that, when read by a processor, cause the processor to perform at least one of requesting via a cloud server at least one patient response to at least one query of at least one health related issue sent to a user equipment of a patient, receiving via the cloud server at least one patient response to the at least one query from the user equipment, determining via the cloud server a review criticality of the at least one patient response based on the at least one health related issue, flagging via the cloud server the at least one patient response as at least one critical flagged response if the review criticality exceeds a predetermined criticality threshold for the at least one health related issue, disclosing via the cloud server to a local health care provider server the at least one critical flagged response in an order of criticality and outreaching via the cloud server to at least one of the local health care provider server and a local health care provider device the at least one patient response if the review criticality indicates a substantial public hazard.

A further example embodiment of the present application provides at least a system, comprising at least one cloud based processor, and at least one memory electrically coupled to the at least one processor and storing an application, the processor performing at least one operation to request via a cloud server at least one patient response to at least one query of at least one health related issue sent to a user equipment of a patient, receive via the cloud server at least one patient response to the at least one query from the user equipment, determine via the cloud server a review criticality of the at least one patient response based on the at least one health related issue, flag via the cloud server the at least one patient response as at least one critical flagged response if the review criticality exceeds a predetermined criticality threshold for the at least one health related issue, disclose via the cloud server to a local health care provider server the at least one critical flagged response in an order of criticality and outreach via the cloud server to at least one of the local health care provider server and a local health care provider device the at least one patient response if the review criticality indicates a substantial public hazard.

### BRIEF DESCRIPTION OF THE DRAWINGS

FIG. 1 illustrates an example of the integrated application platform according to example embodiments.

FIG. 2 illustrates a network configuration of the third party participants of the integrated application according to example embodiments.

FIG. 3 illustrates a user smartphone interface of an example treatment plan according to example embodiments.

FIG. 4A illustrates an example setup configuration for a new course of treatment according to example embodiments.

FIG. 4B illustrates an example database entry for the new course of treatment according to example embodiments.

FIG. 4C illustrates a flow diagram configuration for the new course of treatment according to example embodiments.

FIG. 4D illustrates an example list of messages for the ongoing course of treatment according to example embodiments.

FIG. 4E illustrates an example setup configuration for various courses of treatment according to example embodiments.

FIG. 4F illustrates an example set of details of an ongoing course of treatment according to example embodiments.

FIG. 4G illustrates an example network configuration of the various third parties involved in the application operation and compliance according to example embodiments.

FIG. 5 illustrates a logic module configured to process the input and output parameters of the application according to example embodiments.

FIG. 6 illustrates an example network entity device configured to store instructions, software, and corresponding hardware for executing the same, according to example embodiments of the present application.

FIG. 7 illustrates a data file defining tags and levels of urgency, according to example embodiments of the present application.

FIG. 8 illustrates a patient reply, according to example embodiments of the present application.

FIG. 9 illustrates an electronic report to a health care provider with alert tags, according to example embodiments of the present application.

FIG. 10 illustrates system architecture, according to example embodiments of the present application.

FIG. 11 illustrates an example back to work report according to example embodiments of the present application.

FIG. 12 illustrates a triaged patient response review, according to example embodiments of the present application.

FIG. 13 illustrates a single patient response report, according to example embodiments of the present application.

FIG. 14 illustrates a clustered single employee report, according to example embodiments of the present application.

FIG. 15 illustrates a clustered group report, according to example embodiments of the present application.

FIG. 16 illustrates a clear channel immediate confirmation, according to example embodiments of the present application.

FIG. 17 illustrates a clear channel patient guidance output, according to example embodiments of the present application.

FIG. 18 illustrates a clear channel set of patient instructions and patient response requests, according to example embodiments of the present application.

FIG. 19 illustrates a clear channel direct patient message, according to example embodiments of the present application.

FIG. 20 illustrates a first method, according to example embodiments of the present application.

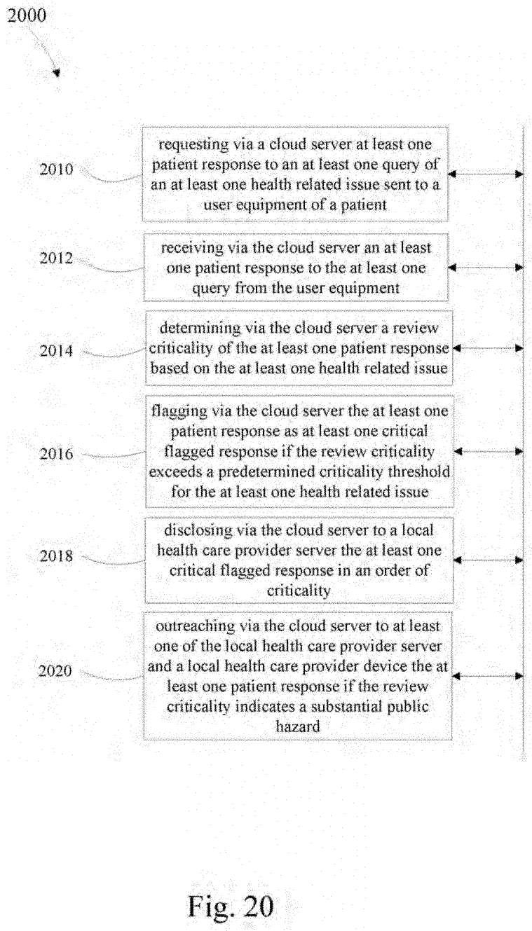

FIG. 21 illustrates a second method, according to example embodiments of the present application.

FIG. 22 illustrates a first non-transitory computer readable medium, according to example embodiments of the present application.

### DETAILED DESCRIPTION

It will be readily understood that the components of the present application, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

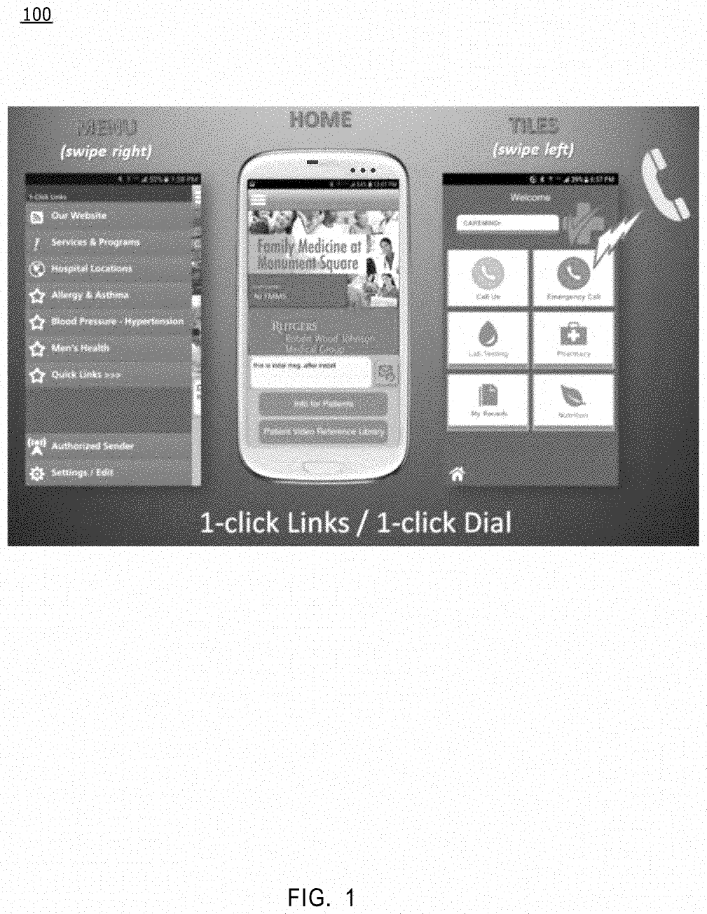

FIG. 1 illustrates an example of the integrated application platform according to example embodiments. Referring to FIG. 1, the configuration 100 includes a menu user interface, a home user interface and a set of option tiles for accessing third party resources, such as test results, emergency concerns, pharmacy information, etc.

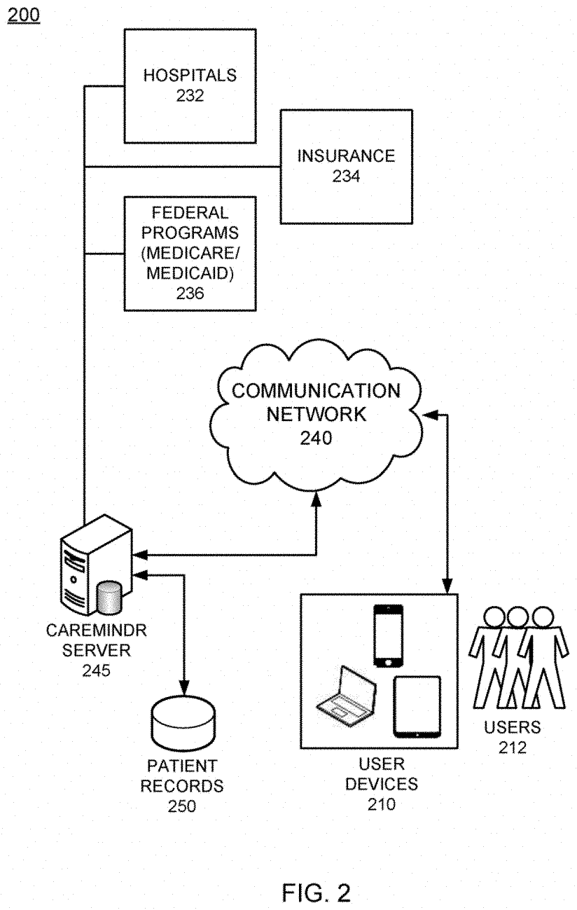

FIG. 2 illustrates a network configuration of the third party participants of the integrated application according to example embodiments. Referring to FIG. 2, the network includes a central server 245 with patient records 250. The information needed to provide treatment plans and other integrated services may require access to hospital and other provider services 232, insurance company information 234, drug providers, federal program administrators 236, etc. The information may be incorporated into any treatment plan or other integrated service model accessed by a user device 210 operated by a user 212. The servers and third party modules may operate on-site or in a cloud network managed by the providers.

Examples of treatment plans and other objectives may include a care management service for assessment of patient medical needs. The system and application may ensure timely receipt of all recommended treatment actions, drugs, third party services and over a designated period of time. Also, referrals to other providers and additional services may provide emergency visits, discharge instructions, nursing facility operations, and home health care functions. In operation, the procedure may begin with the medical treatment provider creating a treatment plan or ‘journey’ for each patient. Each journey is generally for a single chronic condition or objective. One patient may have multiple journeys integrated into a single application. Also, the journeys may originate from various different providers and service entities. The journey will provide the health care provider with biometric, objective and subjective data to enable evidence-based medical decisions. As an example, the biometric data may be glucometer data collected from a blue tooth enabled device and made available to the physician, objective data such as whether the patient visited an emergency room or hospital and subjective data such as how the patient is feeling.

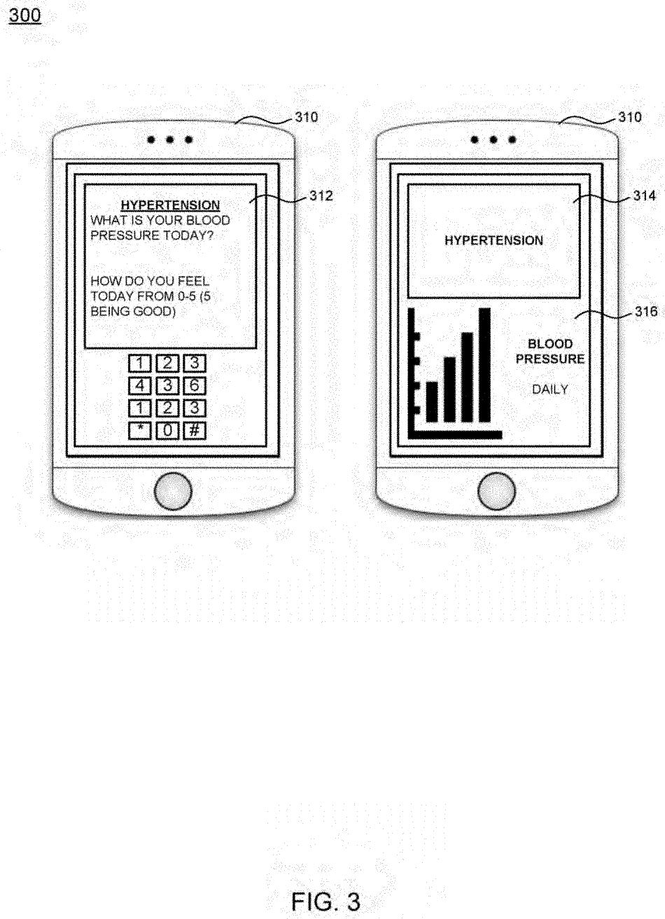

FIG. 3 illustrates a user smartphone interface of an example treatment plan according to example embodiments. Referring to FIG. 3, the journey for “hypertension” may have been created or modified by a patient doctor and may include an interface 300 with a smartphone device 310 and a screen option configuration providing questions 312, information about the treatment, reminders and other functions. The example in FIG. 3 provides for a set of questions 312 and a journey topic 314 along with a graph of blood pressure records 316 as measured over time from various interactions.

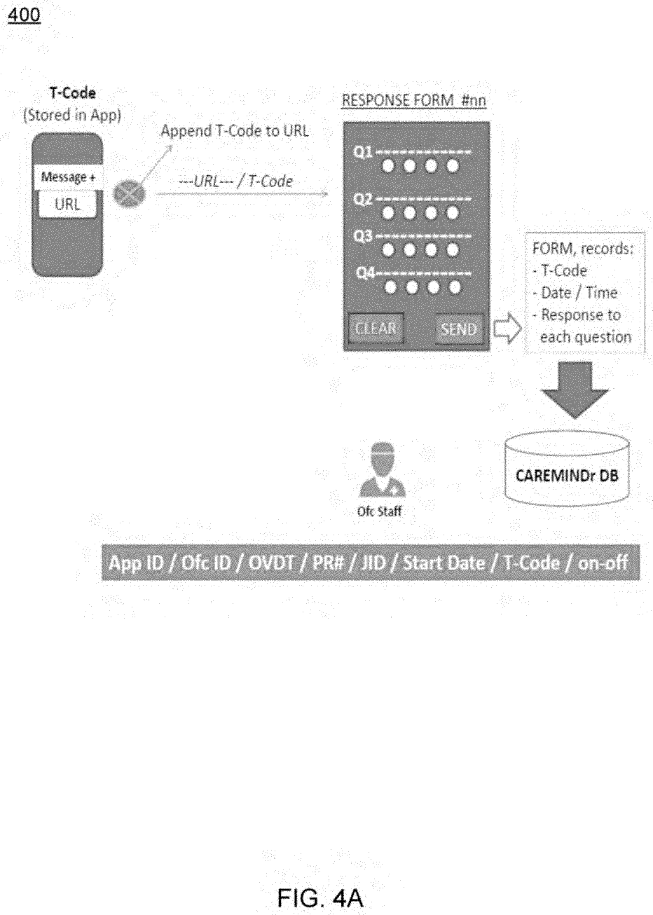

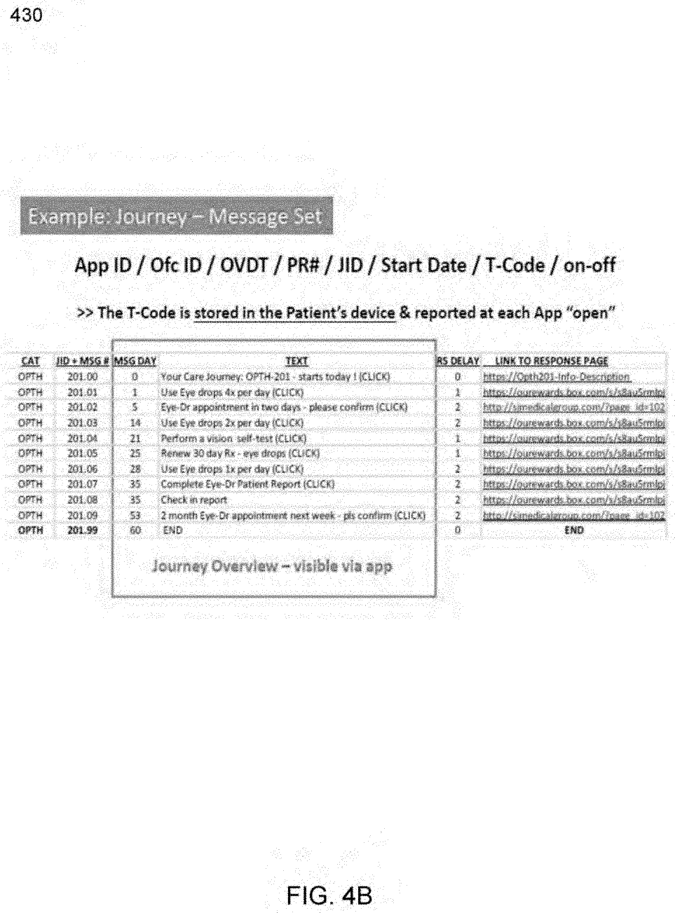

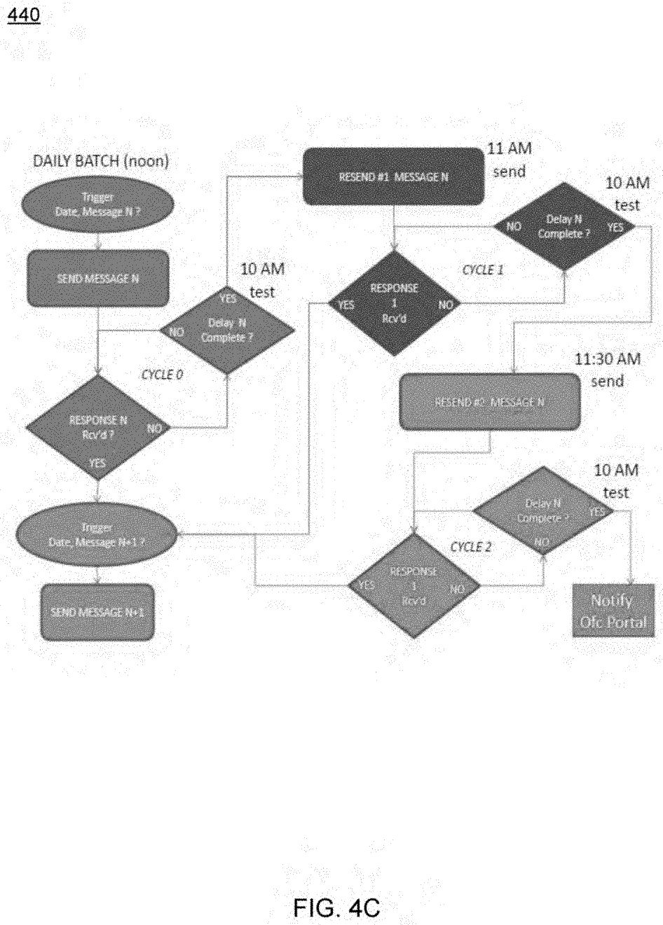

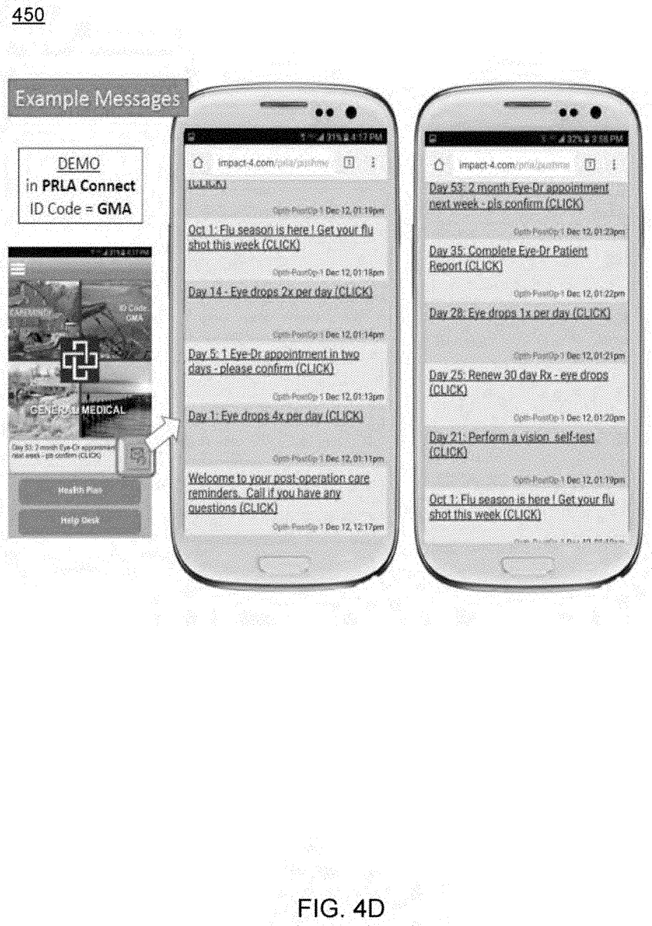

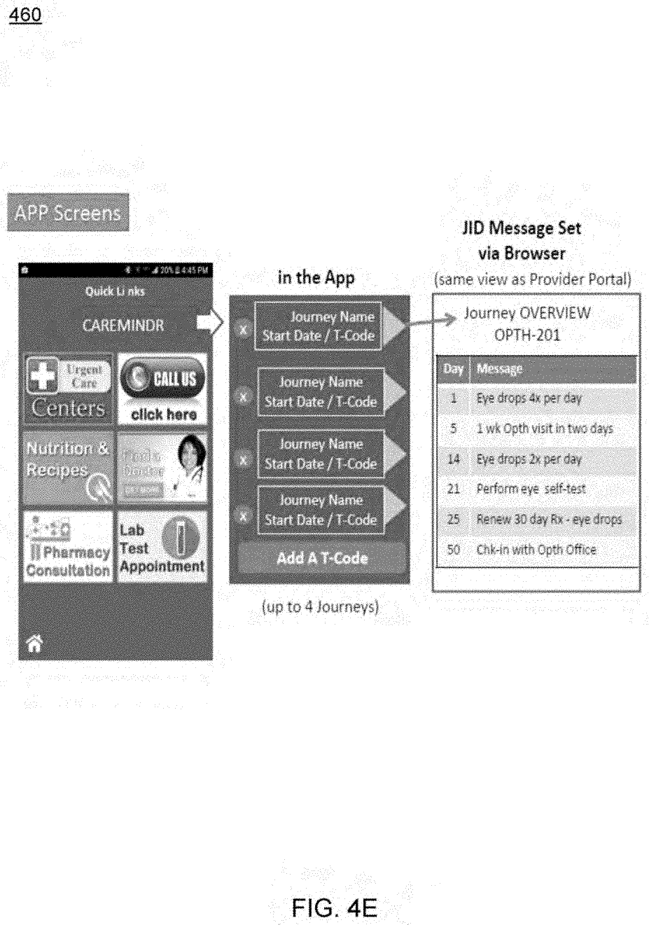

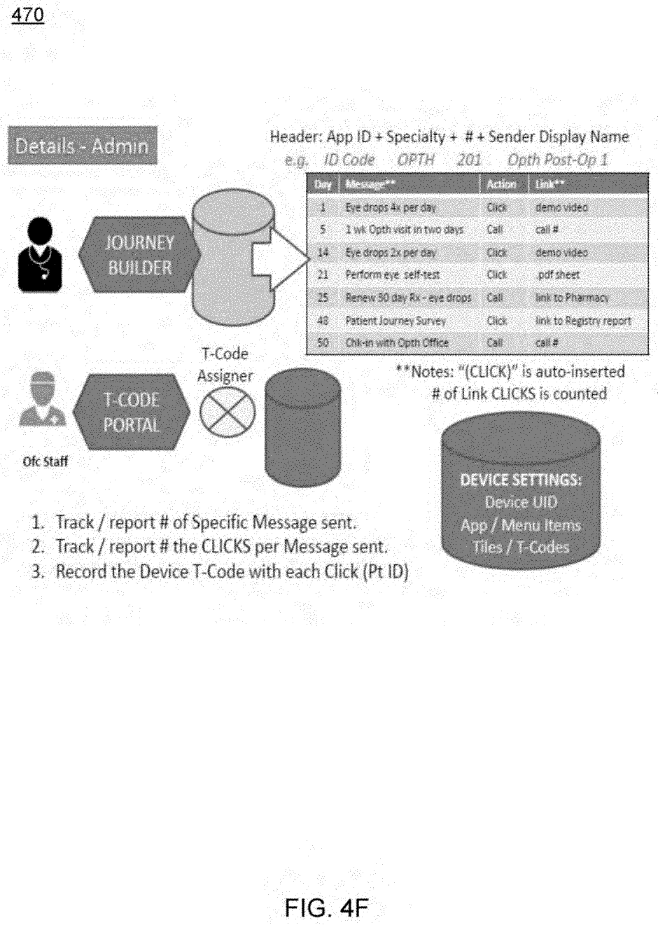

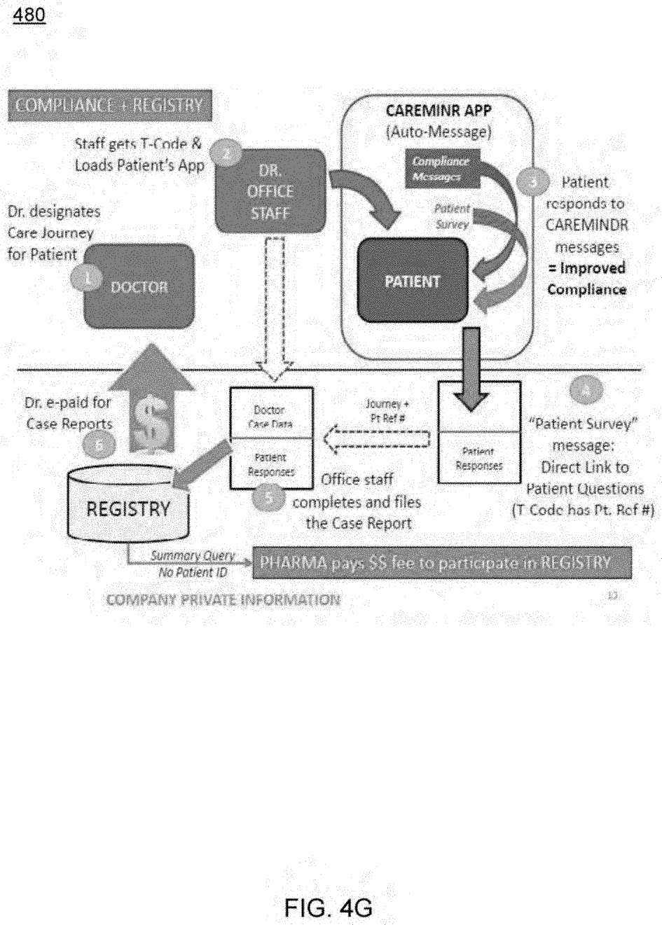

FIG. 4A illustrates an example setup configuration for a new course of treatment according to example embodiments. Referring to FIG. 4A, the illustration 400 includes the basic setup functions of linking a particular journey T-code (unique code) to the message and/or universal resource locator (URL) to link the application of the user to a customized template, such as a response form, questionnaire, etc. The unique T-code, date, time, response, and other records for each instance may be stored in a patient record managed by the application system database.

FIG. 4B illustrates an example database entry for the new course of treatment according to example embodiments. Referring to FIG. 4B, the example configuration 430 includes a database entry of messages which are organized by a category, in this case ophthalmology, and with a message content, including a link to a response page. The context and add-ons of a particular message may be customized based on a preferred layout or a default layout.

FIG. 4C illustrates a flow diagram 440 configuration for the new course of treatment according to example embodiments. Referring to FIG. 4C, the flow diagram includes a daily batch of messages which are setup to be delivered to one or more assigned patients. The process begins with a trigger to send a message, such as a matured date or time. The process then continues to deliver additional messages once confirmation of delivery is made. If the message is delayed or the response required is not received, the message may be resent as a late message requiring immediate attention. The process may continue to cycle to identify whether any messages are outstanding or have not been confirmed.

FIG. 4D illustrates an example list of messages for the ongoing course of treatment according to example embodiments. Referring to FIG. 4D, in this illustration 450, the various messages intended for a particular patient are illustrated by date. FIG. 4E illustrates an example setup configuration for various courses of treatment according to example embodiments. Referring to FIG. 4E, the configuration 460 includes a menu of options along with a set of potential journeys the user may be assigned to manage the ongoing health care treatment plans for that user. The overview of treatment options and dates are included for reference purposes.

FIG. 4F illustrates an example set of details of an ongoing course of treatment according to example embodiments. Referring to FIG. 4F, the details of the administrator are shown to include a journey builder function based on certain parameters, such as an identification code, specialty, a number and a sender name. The number of messages, responses and actions are recorded to demonstrate the user's interaction with the application and the specific treatment plan(s).

FIG. 4G illustrates an example network configuration of the various third parties involved in the application operation and compliance according to example embodiments. Referring to FIG. 4G, the large-scale network of communications among the integrated platform 480 demonstrates the process initiating with the doctor's office establishing a journey for the patient and assigning a T-code (unique code). The patient's responses are identified along with links and references to third party message links and other information sources.

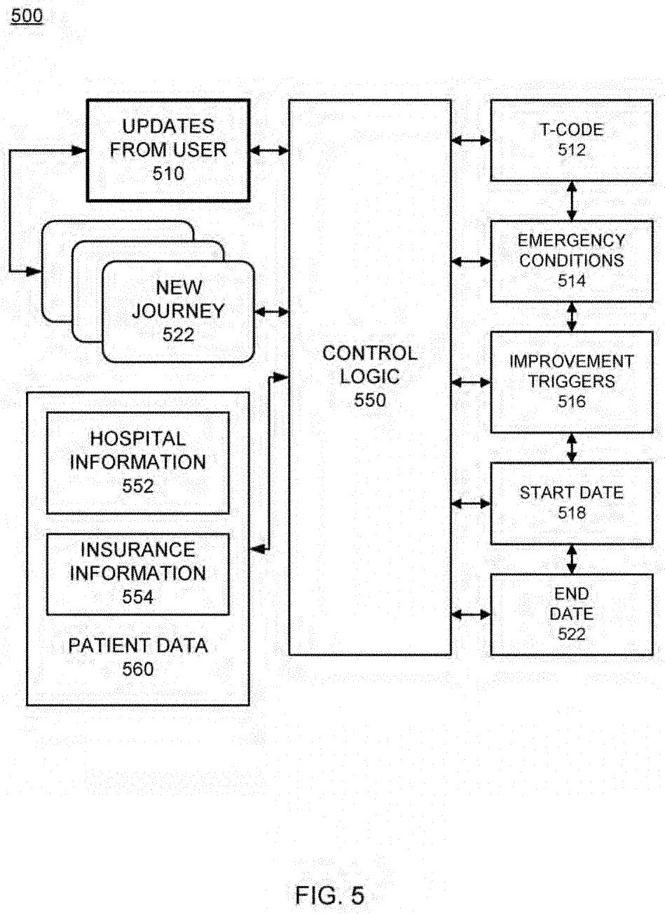

FIG. 5 illustrates a logic module configured to process the input and output parameters of the application according to example embodiments. Referring to FIG. 5, the control logic platform 500 includes a control logic unit 555, such as a processor or other processing entity that may receive updates from a user 510, new journey information 522 and/or patient data 560 including hospital 552, insurance 554, and other information. The logic may be configured to identify and link the unique T-code 512, emergency conditions 514, improvement triggers 516 for optimal changes to the treatment plan, along with dates 518 and new journey information 522 to perform the treatment plan.

In addition, while the term “message” has been used in the description of embodiments of the present application, the application may be applied to many types of network data, such as, packet, frame, datagram, etc. For purposes of this application, the term “message” also includes packet, frame, datagram, and any equivalents thereof. Furthermore, while certain types of messages and signaling are depicted in exemplary embodiments of the application, the application is not limited to a certain type of message, and the application is not limited to a certain type of signaling.

According to example embodiments, a user device, such as a smartphone, cellular phone, tablet device, laptop or other computing device with a memory and processor, may communicate with another computing device and/or a server to provide an integrated communication platform.

Example embodiments provide a computer system programmed to use automated messaging from medical offices to specific patients. The application is not limited to medical procedures and functions and may be used with other configurations for various purposes and services benefitting the end user. Example embodiments include three main computer systems, which work together in an integrated manner including a management platform that controls setup, functionality, activity reporting, and messaging credentials for the users. An administrative platform which the doctor and doctor's office can access via the internet, and a mobile application that a patient can download into a mobile computer device such as a smartphone or tablet. The management platform acts as the nexus of the system sending outgoing messages on behalf of the health care provider and forwarding patient responses to the health care provider's administrative platform. The medical office may have a specific identification that is stored within the management platform.

The integrated platform provides a way of checking-in with a patient at prescribed intervals during times between office visits and when undergoing certain treatment that the doctor is providing or overseeing for the patient. The patient dialog may gather relevant information about the status of the patient's conditions or recovery and can be modified or tailored to specifically meet the dialog requirements of the treating physician. Once initiated by the doctor's office, the application operates in an autonomous manner by delivering messages to the patient to prompt responses if needed. The application functions are monitored to assure that the patient replies to the information requests from the doctor, otherwise a no-response alert is sent to the doctor's office. The interactions are recorded and time-stamped, providing an auditable record of the dialog, suitable for insurance billing purposes. The application can also support biometric information from devices that measure certain body functions, such as diabetes glucometers, or blood pressure cuffs, or any sensory readable health care metric. The application may also create a longitudinal record of information for the patient to illustrate week-to-week trends.

Response Alert Tags

As has been stated earlier, this method and system is utilized when a patient visits a health care provider for an illness/condition which is diagnosed and treated. The treatment occurs over a period of time and is referred to as a journey. The system tracks a patient's progress along the journey for that illness or condition and solicits health information from the patient at clinically-relevant intervals, across an extended time period to enable evidence based medicine. The specific information sought, the intervals, and the time period duration apply to specific conditions or illnesses for which a specific patient is being treated.

This solicitation for patient information may take the form of queries sent to the patient for information, when the responses to those queries are delivered to the patient's health care provider (e.g. physician). The patient's journey may have a number of waypoints occurring at the clinically-relevant intervals. The responses to the queries at these waypoints are meant to determine a patient's progress and status and to present to the health care provider evidence upon which to conduct evidence-based medicine. The responses are collected by the system and measured against historical norms for the patient and/or expected answers for similar patients on similar journeys.

In the event of an unexpected response to a query at that waypoint, the response is treated as notable. Notable events may be considered non-urgent or may be considered urgent or emergent. This divergence from the expected response outcome is graded for severity or urgency. If the severity or urgency of the response exceeds a predetermined threshold for that patient for that journey for that illness or condition at that waypoint, an urgent tag is created and sent to the health care provider. The grading may be one of an immediate medical action advisory, a follow-up advisory and a medical history review advisory.

The information requested in the query is sent in a structured format to allow ease in answering and the response data is delivered to the health care provider in a structured data format to facilitate ease in analysis and trend detection.

The response alert tag is a feature that “tags” certain responses provided by the patient as information that requires follow-up or special notice by the patient's health care provider. The tag may indicate a level of severity or urgency, thus alerting the provider to information that may need immediate medical action, additional follow-up with the patient or a specific review of the patient's medical history.

The tag may be communicated to the provider through multiple channels depending upon circumstance and urgency and in an immediate manner or in a weekly aggregated format depending in part upon urgency.

Work flow instructions may be electronically linked to a tag, so that the specific health care provider that reviews the data will have guidance about the actions to be taken when a tag appears and any escalation of clinical review that might be appropriate.

Each patient for each illness or condition is interacted with by the system at intervals which are relevant to that illness or condition and the queries are sent to determine the patient's progress or status. The received response to the query is measured against an expected response, and anomalies or offsets are noted. If these response anomalies or offsets are larger than a predetermined amount, an urgent or severe issue may need to be addressed. Thus the response is tagged as an urgent tagged response and may be sent utilizing a priority delivery schedule, a priority delivery indicia on the response and may be made to a priority delivery list determined by the health care provider. The response may be tagged as non-urgent if the determined urgency level does not meet the predetermined urgency threshold of the patient for the health related issue.

The structured format allows an overlap of queries so that the patient is not answering multiple identical queries at any one point in time. Additionally, the structured format allows the data to be collected and logged in a structured format and assembled for future review both by the practitioner and the patient to determine trends.

In one example a method, includes requesting via a cloud based system from a patient response to a query and receiving the response to the health related query, determining an urgency level of the response based on the patient health related issue and tagging the response as an urgent tagged response if the determined urgency level exceeds a predetermined urgency threshold of the patient for the health related issue.

The method also includes providing the urgent tagged response to the health provider, where the urgent tagged response may be sent utilizing a priority delivery schedule, a priority delivery indicia for the response and may be made to a priority delivery list.

The method may also include tagging the response as non-urgent if the determined urgency level does not meet the predetermined urgency threshold of the patient for the health related issue.

If the determined urgency level of the response is such that it rises to the level of a medical emergency, then the primary care physician may be immediately notified as well as emergency services such as 911 and if deemed appropriate, dispatched to the location identified either by the patient or gathered from a location indicator in his mobile device. If the response is deemed critical, in situations where the primary physician is not immediately available, an emergency medical specialist may be placed in active direct communication with the patient. The system would make available to the first responder the query and response to provide context for the escalation.

The response may be graded as to the tagged urgency level of the response, where the grading is at least one of an immediate medical action advisory, a follow-up advisory and a medical history review advisory. A follow-on query may be sent based on the urgent tagged response to give the provider context to the urgent tagged response. As an example, if the patient responds that they have been to the emergency room (ER) that may trigger another set of queries about the ER visit to add context to the response. This second set of queries may determine whether the ER visit was related to conditions or illnesses related to the journey, or whether visit was for a condition unrelated to the journey, but still of interest to the health care provider.

In another example a cloud based system links user equipment and a health care provider server. The cloud based system requests a response to a query from a patient pertaining to a health related issue, receives the response to the query and determines an urgency level of the response based on the patient health related issue. The system also tags the response as an urgent tagged response if the determined urgency level exceeds a predetermined urgency threshold of the patient for the health related issue and provides the urgent tagged response to health provider.

The cloud based system may receive via the user equipment a sensor signal provided by a medical device in response to the query. The medical device may be a blood pressure monitor, a glucometer, a pulse meter, a continuous positive airway pressure device, a heart monitor, an implanted medical device and the like.

The cloud based system may receive via the user equipment an audio or text message indicating a medical distress condition in response to the query or may overhear the patient indicating a medical distress condition in conversations or texts in an unsolicited message.

The system may also interpret patient actions in regards to patient historical norms, such as, if the patient is overheard slurring his speech, he may be having a stroke, or if he is discussing that he has pressure in his chest or his left arm is numb, he may be having a heart attack. At this point the system may connect him directly to a medical specialist and take other appropriate action, such as determining his location and dispatching emergency services.

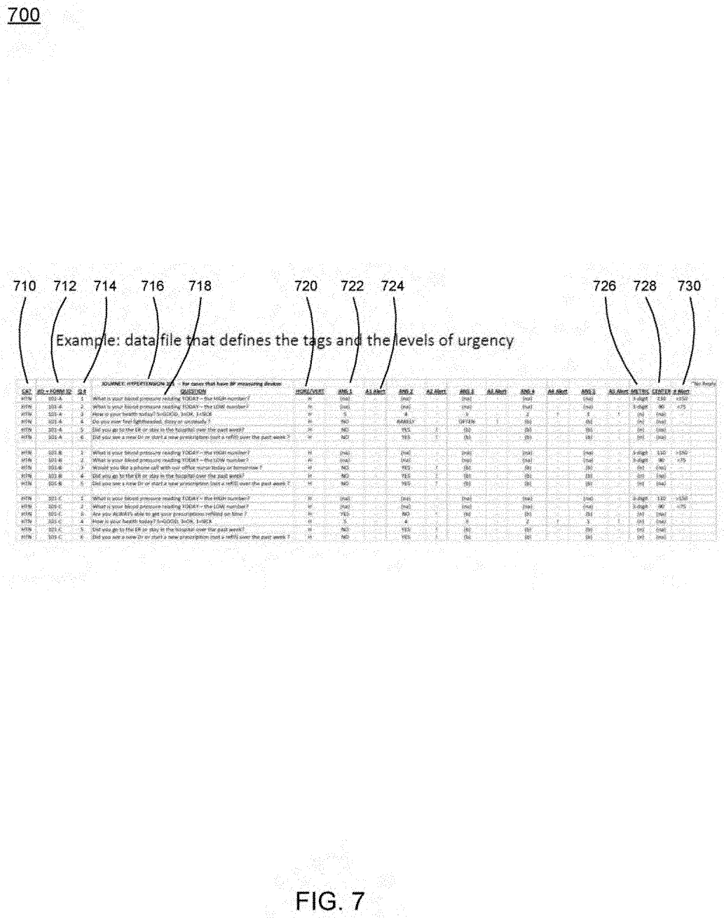

FIG. 7 depicts an example data file 700 that defines the tags and levels of urgency. The parts of the data file include a category of question 710, the journey ID and form ID 712, a question number 714, a patient journey for that specific illness or condition 716 and questions associated with that journey 718. The data layouts may be shown horizontally or vertically 720 which allows viewing of individual answers to queries. The historical and current query response 722 and the determined alert 724 are shown, which in a historical context allows detection of trends. The data output of the metric 726 may be numerical, yes or no, and the like, a median query answer 728 for that patient or that condition and an alert threshold 730 which may be modified by the health care profession are shown.

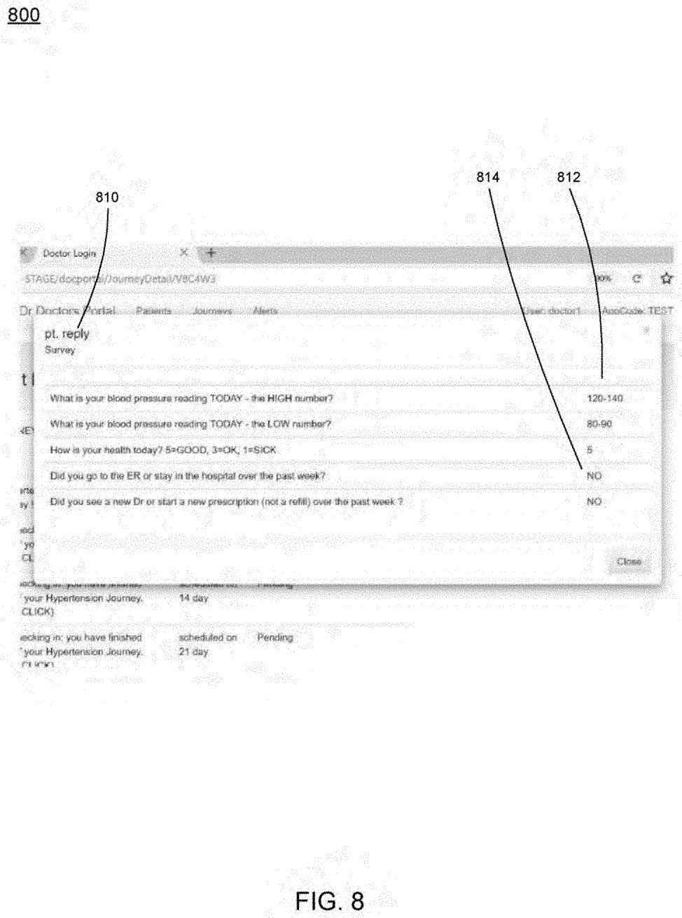

FIG. 8 depicts a patient survey reply 810 having a band of expected replies for blood pressure 812, how the patient feels and whether he went to the emergency room, hospital 814 or has started a new prescription.

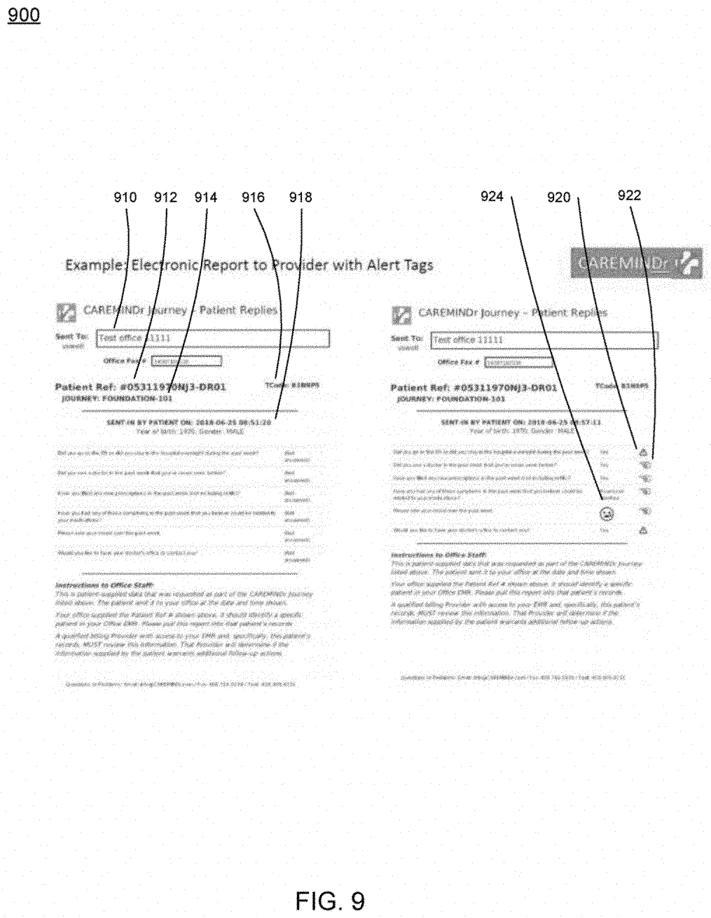

FIG. 9 depicts both a non-responsive and responsive reply set 900. The recipient 910, patient reference number 912, journey ID 914, unique T-Code 916 and timestamp 918 are depicted in the reply. A responsive report having alerts indicates an urgent issue 920, a follow up item 922 and an emoticon 924 indicating a patient feeling is shown.

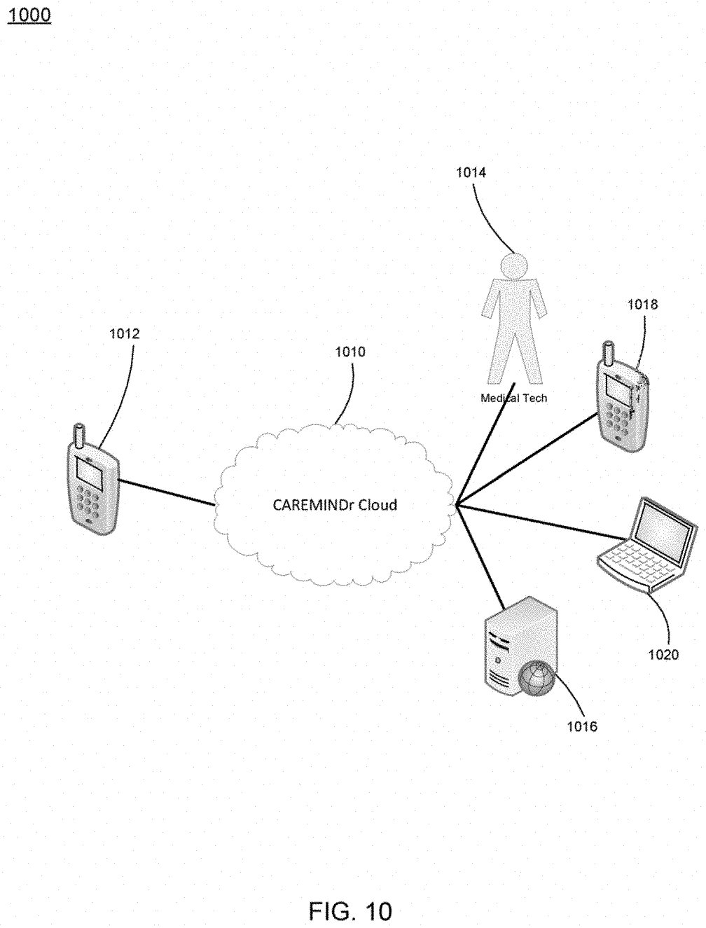

FIG. 10 depicts an example 1000 of communication associated with the alert tag. The cloud based system 1010 sends a request with queries to the patient's communication device 1012 which the patient fills out and returns. The cloud based system 1010 reviews the response and determines whether there are urgent or emergency issues and sends an urgent tagged response to the health care provider.

If there is an emergency issue the cloud based system may contact or place the patient in contact with a medical technician 1014 in addition to notifying the health care provider by means of the health care provider's server 1016, the cloud based system may issue a text or message to the health care provider. The communication route from the health care provider may be by means of mobile device 1018, computer 1020 or the like. The cloud based system may directly connect the patient via to the patient's communication device 1012 to the health care provider under appropriate circumstances. Non-urgent issues are sent to the health care provider for later review.

A second example method of alert tagging, may include requesting a patient response to a message including a query of a health related issue and receiving the patient response to the query. The method then provides determining an urgency level of the patient response based on the health related issue, tagging the patient response as an urgent tagged response if the determined urgency level exceeds a predetermined urgency threshold of the patient for the health related issue and providing the request and the urgent tagged response to a health care provider. The method may also include proposing a set of work flow instructions linked to an urgent or non-urgent tagged response and presenting a clinical escalation review advisory if the response is tagged as an urgent tagged response.

A first example method of alert tagging, may include, selecting a treatment plan for a patient comprising a set of treatment information, linking an application identifier and a T-code identifier to the treatment plan and launching a treatment plan application. The method further includes retrieving the set of treatment information, populating the treatment plan application with the set of treatment information and triggering a message dispatch in accordance with the treatment plan. The message dispatch includes a query to a health related issue to determine a patient status and the system receives a patient response to the message. The method then provides determining an urgency level of the patient response based on the health related issue, tagging the patient response as an urgent tagged response if the determined urgency level exceeds a predetermined urgency threshold of the patient for the health related issue and providing the request and the urgent tagged response to a health care provider. The method may also include proposing a set of work flow instructions linked to an urgent or non-urgent tagged response and presenting a clinical escalation review advisory if the response is tagged as an urgent tagged response.

With respect to the timing of patient responses, the first example method may also include, awaiting the patient response to the message for a late response duration and categorizing the patient response if the patient response is received within the late response duration. If the patient response is not received within the late response duration the method further comprises sending a duplicate message and flagging the patient response as non-responsive if the patient response to the duplicate message is not received within a second late response duration.

The timing of the message dispatches associated with the treatment plan is partly governed by a trigger table. The method may include loading the trigger table having a set of trigger dates based on the treatment plan where the message dispatch is sent according to the set of trigger dates. The method may further include receiving a message start date and receiving an initialization message from a patient mobile device to initiate the treatment plan and to initialize the set of trigger dates in the trigger table.

A first example non-transitory computer readable medium comprising instructions associated with alert tagging of responses that, when read by a processor, cause the processor to perform; linking a user equipment and a health care provider server, requesting from a patient pertaining to a health related issue a response to a query and receiving the response to the query. The processor then determines an urgency level of the response based on the patient health related issue, tags the response as an urgent tagged response if the determined urgency level exceeds a predetermined urgency threshold of the patient for the health related issue and provides the request and the urgent tagged response a health care provider.

Successful patient to health care communication methods will by virtue of their success in getting patients to openly communicate, generate a large volume of communicated health data. Typically, a large percentage of the communicated health data will be uneventful and non-critical. This is especially true if the health care system is communicating with a patient in a situation in which the health care professional wants to ensure that the patient is not going to work sick and possibly infecting others. This large volume of back and forth communication benefits from a system providing a triaged review. The cloud server understands the patient health related issues, proposes questions and reviews the patient responses. If the patient response indicates that there is no actionable issue, or if the response is of a low-criticality nature, then it is triage reviewed as such. If, however, the patient response indicates a critical response based on the health related issue, the response is flagged as a critical flagged response. This review criticality is judged within a spectrum for the health related issue. This triaged critically flagged response may also be judged with respect to external factors such as within the context of an epidemic or pandemic, where seemingly non-actionable responses such as “have you been in contact with someone who has been coughing” may help prevent the spread of a disease.

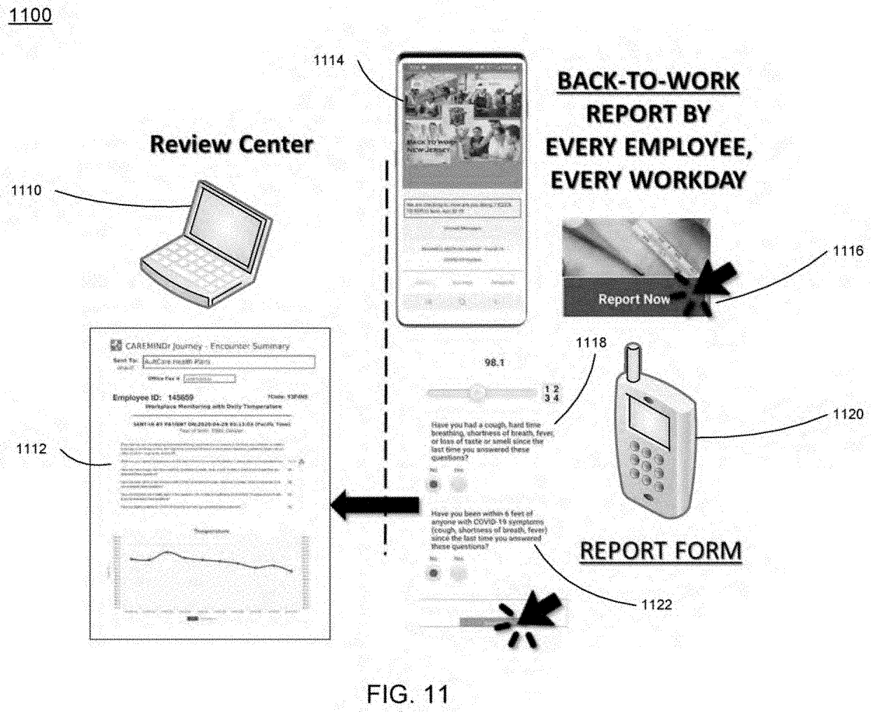

FIG. 11 depicts an example back to work report 1100. In this report a set of questions 1118 and 1122 are sent via a cloud server to the patient user equipment 1120. The patient response data is submitted from the patient user equipment 1120 to the cloud server where the data for the employee 1112 is reviewed by the cloud server and the review center 1110. The patient user equipment 1114 begins the reporting process by the patient user equipment sensing the pressing of the “REPORT NOW” button 1116 on the mobile device.

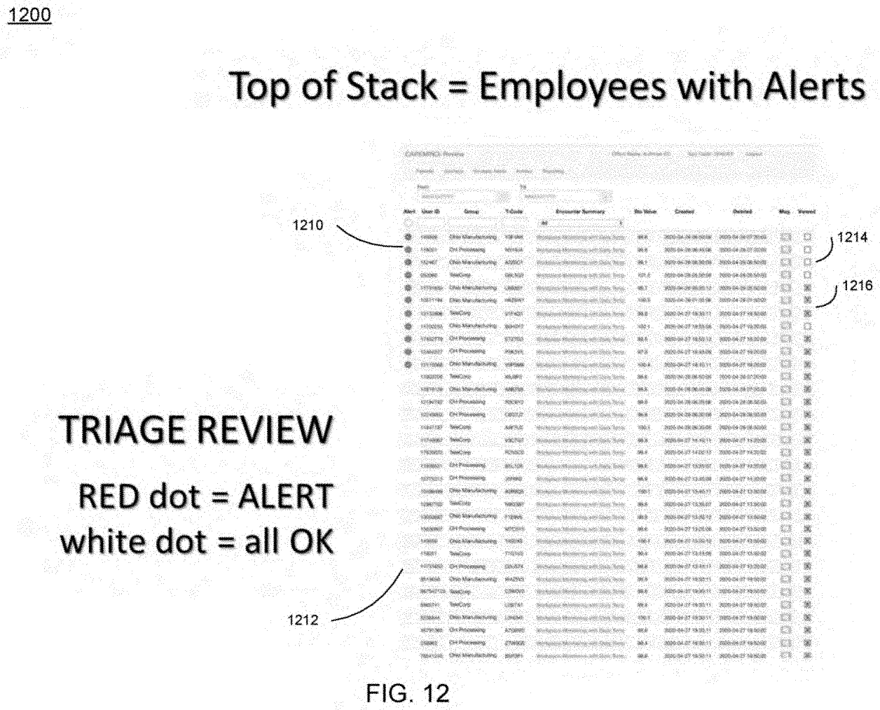

FIG. 12 depicts an example triaged patient response review 1200. In this example report, the cloud server reviews the patient responses and those that are deemed critical responses 1210 are indicated critical and those that do not meet the threshold for criticality are not critically indicated. Also, within this example, those patient responses that have been reviewed are marked reviewed 1216 and those patient responses that have not been reviewed 1214 are left blank.

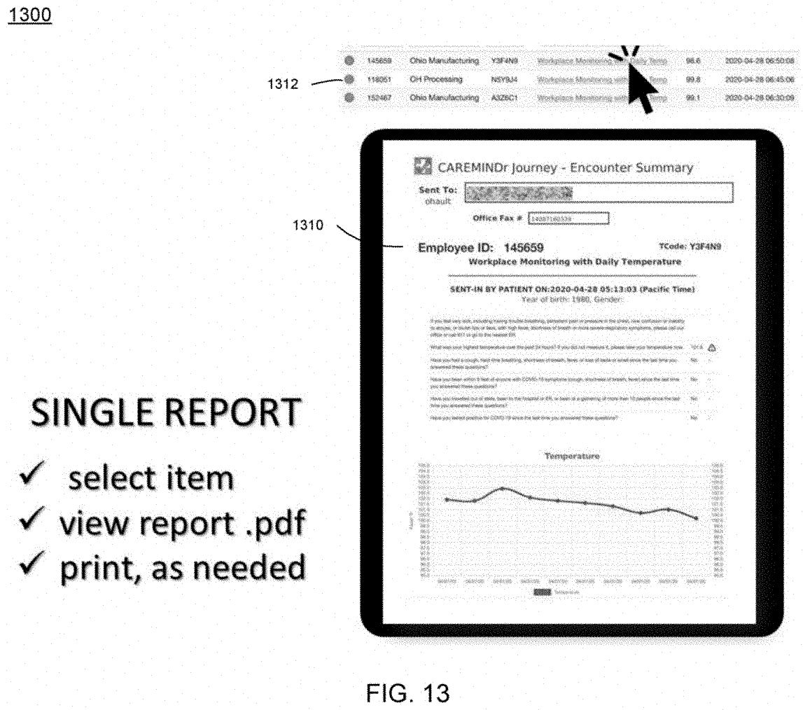

FIG. 13 depicts an example of a single patient report 1300. The patient response has been flagged by the system as being critical 1312, and pressing the hyperlink pulls up the patient report 1310 that is associated with the patient critical response.

The clustering of critical responses may allow the system to be able to detect nascent outbreaks and allow intervention before the outbreak spreads. Each patient response is pre-reviewed by the system to determine its potential effect on the patient and on those in the patient's immediate environment. As an example, if a patient presents a fever of 100.6 and others of the patient's immediate family, or neighborhood or workplace start exhibiting the same condition, the critical response may indicate a nascent outbreak cell. The timely intervention of a nascent outbreak may prevent a wider infection to the family, neighborhood or workplace.

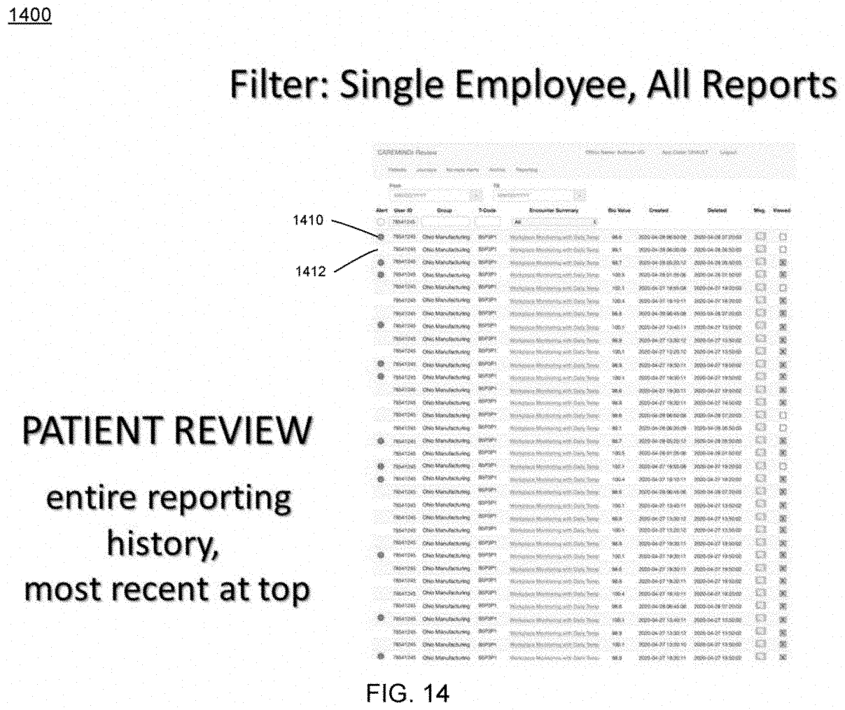

FIG. 14 depicts an example clustered single employee report 1400 generated by the cloud server. The clustering filter allows the health care provider to concentrate on a single employee and review their responses with respect to time. The critical response indicator 1410 indicates a response that the cloud server determines rises above a predetermined threshold for criticality. The non-critical response indicator 1412 indicates those responses that do not rise to the threshold of criticality. The ability to filter for a single employee allows a health care provider to quickly determine how many and how often the critical responses are determined. This functionality allows the cloud server and the health care provider to quickly determine whether a problem is getting worse.

If the patient sends a response or a response in context with other data that indicates that the patient may be a “patient zero” of a localized outbreak, a potential warning may be sent to the health care device, the patient user equipment and or the employer user equipment. The review therefore is a review of the patient response, but may be interpreted by the system within a micro-environment of the patient that includes additional data from the patient user equipment and others within the patient's immediate locale within a period of time.

The system may also have the ability to cluster the patient response with respect to time for that patient and with respect to other patient responses with time, so that a progression may be determined for both the patient and the patient environments such as family, neighborhood, region and workplace. This clustering in time may allow a potential spread pattern to be determined. If a spread pattern may be determined, then it may be possible to contain the movement of a condition or disease based on probable patient interactions.

In a larger context, those patients which take part in the health care response system described by this disclosure are a sampling of a larger group. The patient is a member of a family, a member of a neighborhood, a member of a work community, a member of a social community, and the like. What affects the patient, may affect additional members of the communities to which the patient belongs. The patient therefore becomes a statistical sample of several communities or groups.

Additionally, other patients within this system also belong to various communities, some of which may overlap with the first patient. What we have therefore are overlapping communities to which the patients may belong.

If the first patient responds in a way indicating the onset of an illness, such as responding “generally, I am feeling tired and my muscles ache” and another patient within a cluster of time also presents in the same manner, this may indicate the onset of an affected group of people. If the communities of these patients overlap, then the risk of a multi vector contagion has also increased. The ability of clustering similar presentations to a time and community or region may prove invaluable to being able to detect the seeds of an outbreak.

For every patient that is capable of responding to the system, there may be multiple others that are outside of the patient reporting system. The system may be able to identify others in community with the affected patients by accessing social media databases that indicate a relationship to the patients involved, community registration databases such as tax registers, postal record databases showing regional confluence, work registers showing professional confluence, etc.

Once additional community members are located, they may be contacted by text, mail, email and the like to indicate that they may be at enhanced risk and may need to seek medical testing or medical help. In this way, clustering in time and by communities has informed the larger group of a potential issue to be aware of and make adjustments for, for the health of that person and others within his or her various communities.

By way of example, suppose we have two patients within a neighborhood of 1000 people that are presenting similar illness patterns within a period of three days. If within this neighborhood of 1000, say 100 are connected to the system. Those within the neighborhood that are part of the system may be queried targeting similar symptoms as the two affected patients. The other 900 people within the neighborhood may be contacted by accessing various databases indicating an association with that neighborhood, such as social media and tax rolls. The other 900 people at risk would be asked to respond back in some way to close the loop and give the system feedback that they had been contacted and understood the enhanced risk.

The system may determine enhanced risk of those within the system by tracking in time and for various communities for which the system has access, similar presentations of symptoms. Once these similar symptoms are recognized by the system and correlated to determine if there are overlapping communities. If overlapping communities are found, those within the system may be queried to determine whether they are also presenting similar symptoms. The nexus of time and overlapping symptoms and communities allows tracing of possible exposure to those highest at risk within the various communities. Those outside of the system, but within the communities, may be traced by accessing various databases indicating association with the affected communities. Contacting those outside of the system and closing the medical loop may help stem the tide of an outbreak.

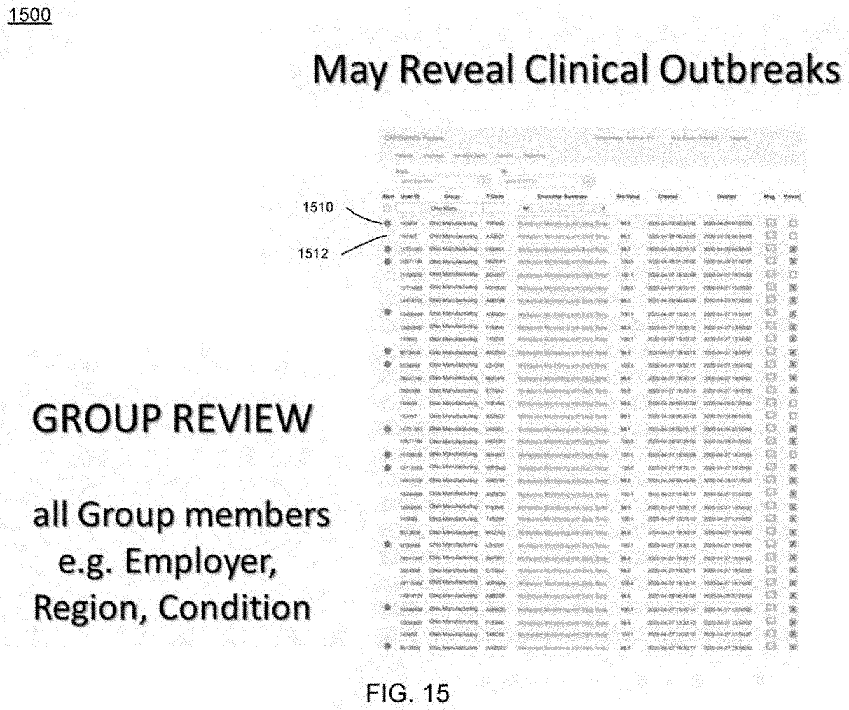

FIG. 15 depicts an example clustered group report 1500. The clustering filter may be set wider in scope than depicted in FIG. 14 in which group members of an employer, region or condition may be consolidated to detect patient critical responses 1510 and non-critical responses 1512. This multiple clustering capability may allow detection of clinical outbreaks.

Comprehension of instructions from the cloud server may mean the difference between a healthy outcome and a potentially hazardous situation for both the patient and others in their immediate environment. The cloud server parses and interprets the patient response to determine whether the patient understands the query and instructions. The patient additionally has the capability to respond by telling the system that one of its queries or instructions was not understood, at which point the system may rephrase the query or instruction for the patient.

Compliance with instructions from the cloud server may be accomplished in one example by response to an additional query. In another example, if the patient is told not to go to work because of enhanced risk and the GPS on the patient user equipment or a local network at the workplace indicates that the patient user equipment is indeed at work, the patient may be considered non-compliant. Non-compliance with respect to the employee's personal health presents a localized risk to the patient, if the patient poses a risk to other employees, non-compliance may be reported to the health care provider device and the employer user equipment. The method may be able to identify and reduce exposure of the patient to other members of the community, by determining the comprehension and or the compliance of the patient.

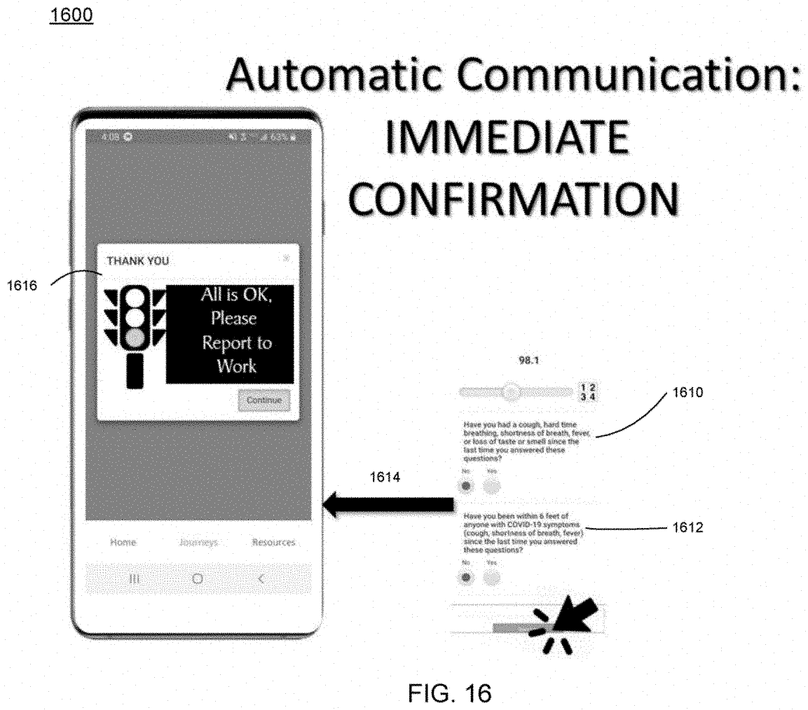

FIG. 16 depicts an example clear channel immediate confirmation 1600, which may be used to answer questions 1610 and 1612 sent 1614 from the cloud server to the patient user equipment 1616. This immediacy allows use for clearing an employee to report to work in a safe manner to prevent the spreading of potential diseases to other employees. Clear channel communication, also termed as direct messaging, will be described in detail later in this specification as system framework messaging.

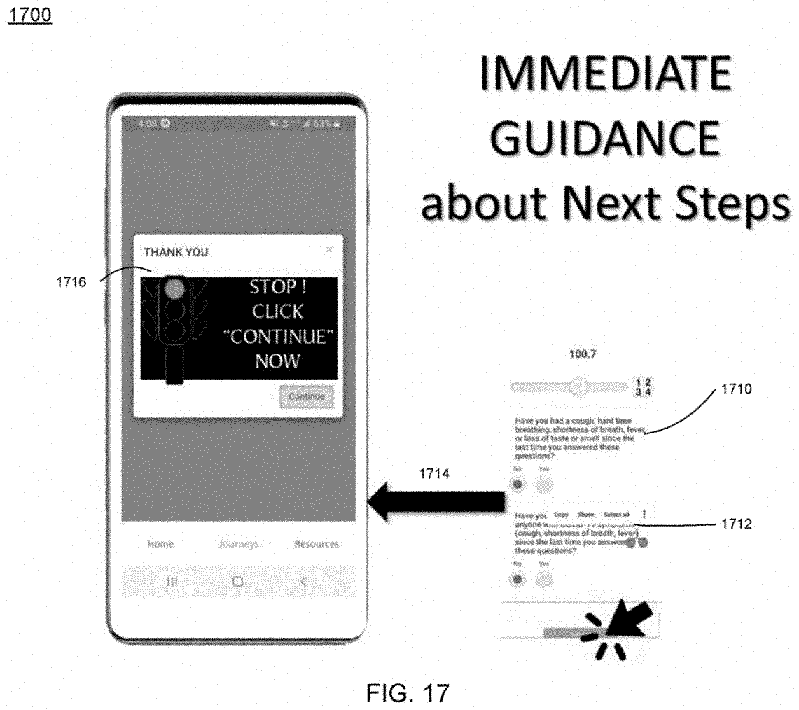

FIG. 17 depicts an example clear patient guidance output 1700. In this example the questions 1710 and 1712 sent 1714 from the cloud server are received at the patient user equipment 1716.

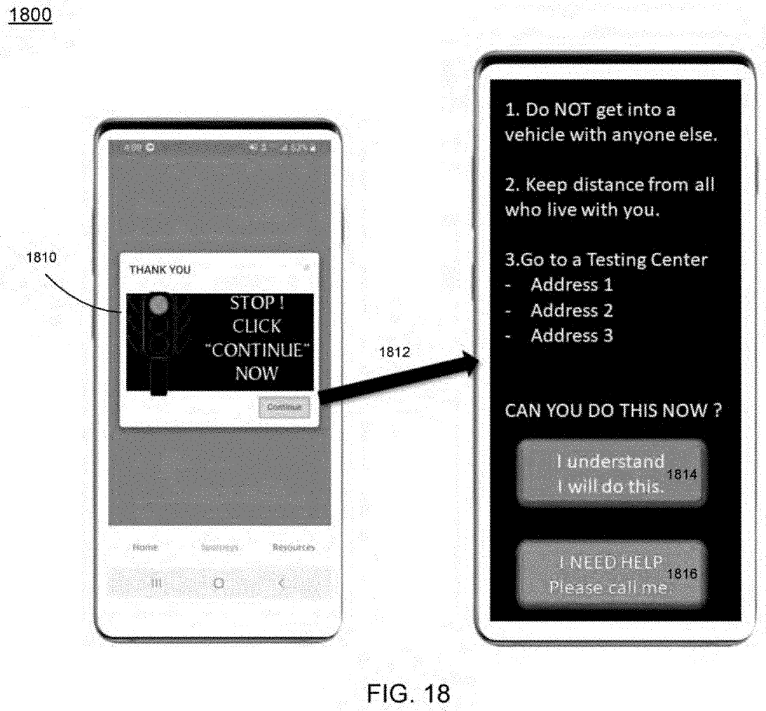

FIG. 18 depicts an example clear channel set of patient instructions and patient response requests 1800. In this example the patient user equipment is instructed to stop and click “CONTINUE” 1810 which then includes a set of patient instructions 1814 and patient may request help 1816 contact information or additional assistance may be sent 1812 from the cloud server.

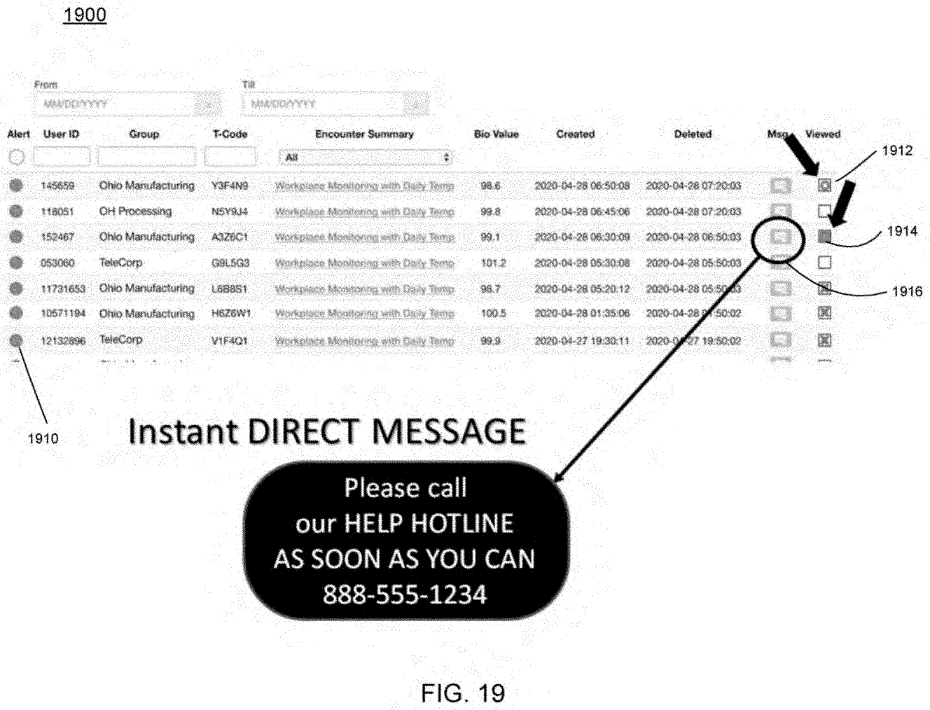

FIG. 19 depicts an example instant direct message 1900 from the cloud server. A direct message in this specification is taken as synonymous with a system framework message, which will be described in detail later in the specification. This example indicates responses that exceed the threshold for critical responses 1910 and indicates those responses that have been reviewed 1912 and those which have not been reviewed 1914. The report also allows a message 1916 be sent utilizing an instant direct message.

A first example method of triaging alert tagged responses includes requesting via a cloud server at least one patient response to at least one query of at least one health related issue sent to a user equipment of a patient and receiving via the cloud server at least one patient response to the at least one query from the user equipment. The method includes determining via the cloud server a review criticality of the at least one patient's response based on the at least one health related issue and flagging via the cloud server the at least one patient response as at least one critical flagged response if the review criticality exceeds a predetermined criticality threshold for the at least one health related issue. The method additionally includes disclosing via the cloud server to a local health care provider server the at least one critical flagged response in an order of criticality and outreaching via the cloud server to at least one of the local health care provider server and a local health care provider device the at least one patient response if the review criticality indicates a substantial public hazard.

Determining an order of criticality for the health care provider may allow increased work flow efficiency. The health care provider in a sense is given a triaged list of the most critical patient responses for review as determined by the system. Those responses that indicate the greatest patient risk or the greatest community risk are placed at the top of the queue for subsequent review and asynchronous communication. This may allow the health care provider to interact and close the loop with those patients that the system considers the highest priority.

The use of the term asynchronous communication is one in which the health care provider does not need to have communication at the same moment in time with the patient. In this way the health care provider may provide input or ask subsequent questions and the patient may answer or review instructions at a time of his choosing. This a-synchronicity of communication allows much more efficient communication between the health care provider and the patient. In this way the health care provider may immediately communicate with the patient. This allows the health care provider to close the communication loop in a more expeditious manner.

The at least one query to the user equipment may coincide with a back-to-work schedule of the patient of a workplace. The method may also include determining via the cloud server a comprehension of the at least one patient response of the workplace to the at least one query sent from the user equipment and or a containment compliance of the at least one patient response of the workplace to a set of health care instructions sent to the user equipment. The method may alert an employer user equipment if the at least one patient response does not indicate the containment compliance of the at least one patient response sent from the user equipment. The method may also determine via the cloud server if a cluster of similar at least one critical flagged responses are detected within the workplace of the patient and or whether the patient repeats similar at least one critical flagged responses. The method may also determine via the cloud server if a cluster of similar at least one critical flagged responses is detected within a group of at least one of a workplace, a region and a condition.

The patent user equipment may also have the capacity to determine whether the patient has fallen, what the patient's temperature is and whether the patient is exhibiting any behaviors that are unusual for the patient, such as frequently sitting down or lying down, or immediately running as if to run to the restroom to vomit or use the bathroom. Small changes in and of themselves may not be an absolute indicator of a problem, but may be noticed by the cloud server in assessing the context of a patient response. Since human communication occurs within a context, the physical responses of the patient may assist in creating a micro-environment from which the patient is communicating with the system.

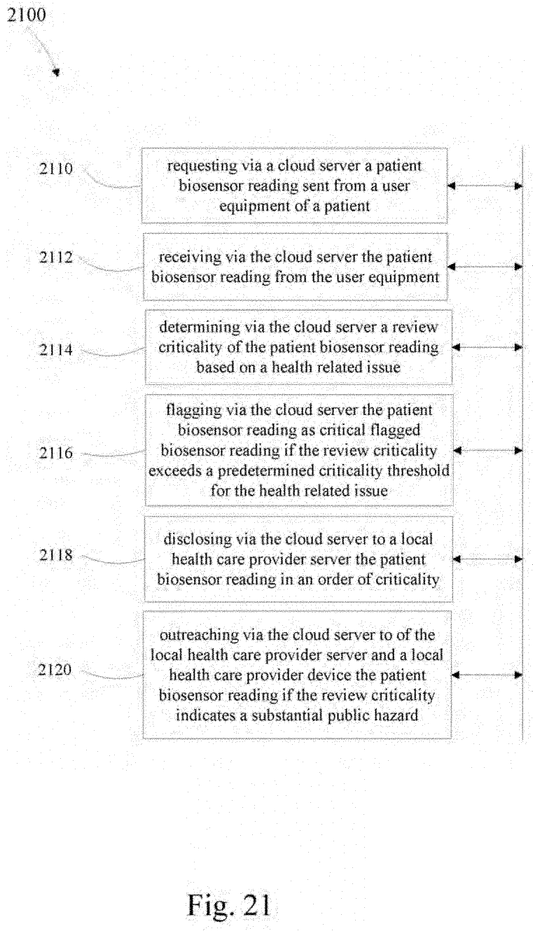

FIG. 21 depicts a second example method of triaging alert tagged responses includes requesting 2110 via a cloud server a patient biosensor reading sent from a user equipment of a patient and receiving 2112 via the cloud server the patient biosensor reading from the user equipment. The method further includes determining 2114 via the cloud server a review criticality of the patient biosensor reading based on a health related issue and flagging 2116 via the cloud server the patient biosensor reading as critical flagged biosensor reading if the review criticality exceeds a predetermined criticality threshold for the health related issue. The method additionally includes disclosing 2118 via the cloud server to a local health care provider server the patient biosensor reading in an order of criticality and outreaching 2120 via the cloud server to of the local health care provider server and a local health care provider device the patient biosensor reading if the review criticality indicates a substantial public hazard.

The patient biosensor reading may coincide with a back-to-work schedule of the patient of a workplace. The patient biosensor reading may indicate a fall of the patient in a non-linear fall pattern, an abnormally high temperature of the patient, an abnormal heart rate pattern of the patient, an abnormal respiration pattern of the patient and or an abnormal movement pattern of the patient. The method may also include determining via the cloud server if a cluster of similar critical flagged biosensor readings is detected within a workplace of the patient, if the patient repeats similar critical flagged biosensor readings, and or is detected within a group of a workplace, a region and a condition.

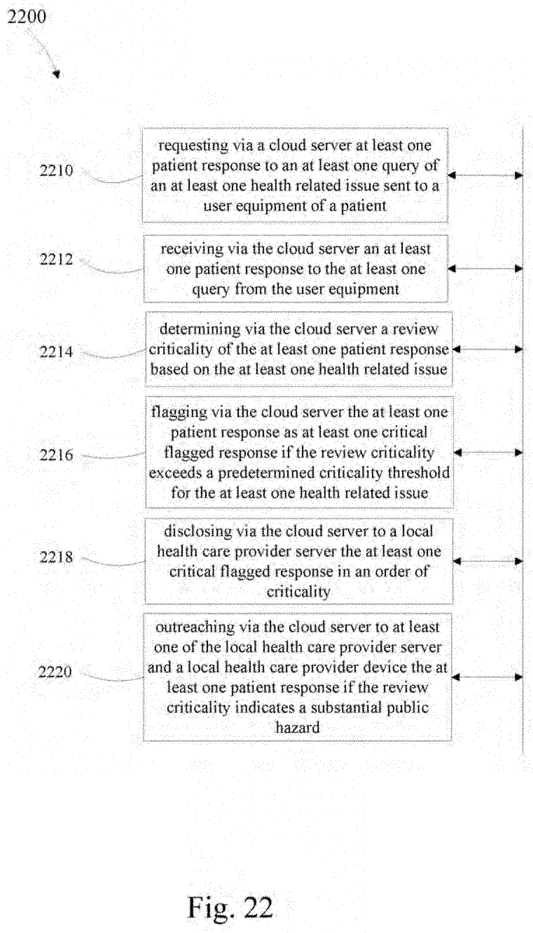

FIG. 22 depicts a first example non-transitory computer readable medium includes instructions associated with triaging alert tagged responses that, when read by a processor, cause the processor to request 2210 via a cloud server at least one patient response to at least one query of at least one health related issue sent to a user equipment of a patient and receive 2212 via the cloud server at least one patient response to the at least one query from the user equipment. The processor determines 2214 via the cloud server a review criticality of the at least one patient response based on the at least one health related issue and flags 2216 via the cloud server the at least one patient response as at least one critical flagged response if the review criticality exceeds a predetermined criticality threshold for the at least one health related issue. The processor also discloses 2218 via the cloud server to a local health care provider server the at least one critical flagged response in an order of criticality and outreaches 2220 via the cloud server to at least one of the local health care provider server and a local health care provider device the at least one patient response if the review criticality indicates a substantial public hazard.

A first example system to triage alert tagged responses includes at least one cloud based processor, and at least one memory electrically coupled to the at least one processor and storing an application, wherein the processor performs operations to request via a cloud server at least one patient response to at least one query of at least one health related issue sent to a user equipment of a patient and receive via the cloud server at least one patient response to the at least one query from the user equipment. The system determines via the cloud server a review criticality of the at least one patient response based on the at least one health related issue and flags via the cloud server the at least one patient response as at least one critical flagged response if the review criticality exceeds a predetermined criticality threshold for the at least one health related issue. The system also discloses via the cloud server to a local health care provider server the at least one critical flagged response in an order of criticality and outreaches via the cloud server to at least one of the local health care provider server and a local health care provider device the at least one patient response if the review criticality indicates a substantial public hazard.

The processor may perform an operation to determine via the cloud server a comprehension of the at least one patient response of the workplace to the at least one query sent from the user equipment, and or determine via the cloud server a comprehension of the at least one patient response of the workplace to the at least one query sent from the user equipment and determine via the cloud server a containment compliance of the at least one patient response of the workplace to a set of health care instructions sent to the user equipment. The processor may perform an operation determine via the cloud server if a cluster of similar at least one critical flagged responses are detected within the workplace of the patient.

Up to 90% of health outcomes are the results of social, behavioral and economic factors. The associated five key social needs such as food insecurity, housing instability, utility needs, transportation needs and experience with interpersonal violence. The key social needs associated with health outcomes are predominantly tied to economic factors. One possible solution to reducing the economic burden of health care is to make communication between a health care provider and a patient as low cost as possible.

The economic state of a patient may have effects on the number of interactions and the seriousness of the health care matter at the time of interaction. In each case for those that are at an economic disadvantage the effects may be to put off health care until a situation that may have been easily manageable becomes a much more serious health care matter. Factors affecting the economic state of an individual or family may be based on socioeconomic factors such as education, job status, family social support, income and community safety and the physical environment such as a residential location, e.g. zip code. This initial set of factors may account for up to half of a healthcare outcome factors.

One example of the system may include an ongoing auto-monitoring portion. The patient may enroll by inputting their user identification, group, initials, year of birth and gender. An auto-survey may be sent to the patient on a prescribed schedule. The auto-survey may result in different sets of guidance's based on the survey response. The auto-survey may ask questions pertaining to race, whether the family's main source of income is based on seasonal or migrant farm work and whether they have been discharged from an armed service branch of the United States. The survey may include the primary language of the patient, how many people live in the household, the housing situation, and whether there is a concern about losing housing, address, educational level, current work situation, insurance and income. The survey may query about the patient's inability to get food, utilities, and medicine or phone services, lack of transportation, interpersonal discussions, stress, whether the patient has spent two nights in incarceration, refugee status, country of origin, physical and emotional safety and fear of a partner or ex-partner. Depending upon the answers of the patient to the auto-monitoring questionnaire, the patient may then be provided with access to information and flyers for available help in accordance with the answers supplied by the patient.

Currently, messaging to a cell phone may comprise a texting charge. One possible solution to reduce this economic burden is to have the cloud server directly message the patient from within the application. This direct messaging may be performed without the patient incurring a charge for the direct message. Direct messaging may in one example be based on communicating through the application based on an embedded peripheral driver within the system and patient user equipment.

In one example, the proposed solution may send a push notification to a user device of a patient, where the user device has the appropriate credentials to access cloud server databases. A structured interactive form has information related to a health related issue of the patient. The structured interactive form is stored in the cloud server database. The structured interactive form may be modified on the user device of the patient and the modification of the structured interactive form is performed on the user device of the patient and is sent to the cloud server. The user fills out the structured interactive form by entering digits or clicking on specific responses. When a reply form is completed, the user will click the “SEND IN” button on the form. This sends the modified data from the reply form to an encounter summary database on the cloud server. The reply form is coded with information indicating that the response is specific to the user device of the patient. In this way, only data pertaining to the masked patient identity and modifications to interactive forms which are not passed back and forth is communicated. This masking and modification communicates very little data.

The proposed solution communicates in a privacy-safe mode with individual users or with groups of users. The users and groups of users of this system may be either patients or parents of patients.

The proposed solution does not utilize texting, the user's phone number, or email. Groupings may be defined by a healthcare organization such as the doctor office, the medical group, the clinic, the insurer, and the like or by a healthcare interest which the user chooses to join such as a pertaining to a condition, a form of treatment, a wellness topic and the like.

The messaging which occurs within the system framework may fall into at least one of two categories, the first being direct user-specific messages and the second being broadcast messages sent to groups to which the user belongs. System framework messaging, i.e., direct messaging, uses a series of database files which are accessed at the various steps by the mobile application installed in the user device of the patient.

A system framework message which is also referred to as direct messaging since messages are transferred directly from database to database and are not actually sent from the cloud server to the user device and vice versa. The messages are viewed via the application on the user device of the patient and the dialog of communication occurs in a structured manner where the user device accesses forms on a remote cloud server. The user device of the patient does not directly connect to the local health care provider server of the doctor with whom they are communicating, but communicates modifications to structured interactive forms that are accessible by the local health care provider server.

In one example, the user device of the patient is prompted to report specific information to the local health care provider server of their doctor's office, regarding a health condition that is being managed by that doctor.

A tracking code is tied to and identifies the user device that was used when the patient initiated their management program, called a journey, as prescribed by their doctor.

Based on an automated schedule that is specific to the patient's journey a push notification may be sent to the user device, alerting the user device that their doctor has sent them a message via the cloud server that needs to be read. This notification is a short set of characters that does not describe the contents of the message. The message may say something such as “YOU HAVE AN IMPORTANT MESSAGE FROM YOUR DOCTOR”.

When the user device senses a click on the “NOTIFICATION” button, the application opens, and in the application's home screen a message may be displayed in the message box such as checking in with you about your diabetes, with an instruction to “PLEASE CLICK”. In the system framework messaging system the home screen is connected to a cloud server. The message box displays the most recent journey message assigned to that user device in the cloud server's unread message database.

The user device may then sense the user clicking the text in the message box, the screen may change from the home screen to view a reply form associated with the message, as it is stored in the journey reply form database.

The structured interactive form may be filled in on the user device where the patient may enter digits and or input specific responses. When the reply form is completed the user device may sense the patient click on the “SEND IN” button of the form. The data from the reply form may be transferred to an encounter summary database on the cloud server. The reply may be coded with journey information indicating that this response is specific to the user device of the patient.

The doctor's office may have a doctor office portal located on the local health care provider server which provides at least two ways to view the encounter summary database, either as patient-specific journey-specific activity or as a consolidated log of the patients' reports via the proposed solution referred to as reviewer.

Utilizing system framework messaging, i.e. direct messaging, the information has been requested, a response created and delivered for viewing by the doctor's office without any large amount of data being transferred from the user device to the cloud server or vice versa.

In a similar manner, other types of messages may be presented to the user device, for example informational viewing, a structured interaction or entering numerical information.

The system framework messaging process, i.e. direct messaging, sends and receives information via interactive forms to move data between databases in the cloud servers. To the user this appears as a request-send-and-receive process, although the interaction is with structured interactive forms which are not as a whole sent or received; only modifications are sent or received.

The system framework messaging process protects personally identifiable information through masking the patient identity. The process does not send a large amount of information from the user device to the cloud server. Rather, devices with the appropriate credentials access the databases in the cloud server so that there is no actual routing of messages.

The proposed system framework messaging does not burden user devices with a heavy computational or communications load. The proposed framework displays interactive screens and transmits a minimal number of keystrokes that consumes very little data on user devices from telecommunications carriers. Therefore, this form of communication is essentially free to the user.

The proposed system framework messaging allows information to be conveyed in a manner that does not rely on elevated verbal or written language skills and thus may be applicable to large numbers of users from varied backgrounds.

Direct messaging may have several effects, one of which is a higher frequency of communication with the patient as the cost obstacle is removed and the second is an increase in the depth of communication between the patient and the health care provider. The removal of the cost impediment may result in more effective communication based on frequency and depth.

A patient perception component of direct messaging may be implemented by a learning system in which the cloud system parses the patient responses and determines the nomenclature that the patient may have difficulty understanding based on patient responses. The ability of the system to parse the patient responses may also be partially based on the co-alignment of patient responses and patient user equipment sensor feedback. In one example, if the patient states that his temperature is 98.6 degrees Fahrenheit and the patient user equipment indicates that the patient actual temperature is 99.4, the system may ask a second question to train the patient to be more forthcoming.

The system has the ability to determine sensor capabilities of the patient user equipment and utilize those sensor capabilities directly in conjunction with direct written communication to expand the data reach of the system. This multi-channel data detection and communication protocol strengthens the written and tactile link to enhance accuracy in parsing communication and verifying the accuracy of the communication proffered by the patient.

An example method of direct messaging includes, issuing a push notification via a cloud server, to a user equipment of a patient and connecting the user equipment of the patient to at least one database that is accessible by the cloud server. The method includes displaying on the user equipment of the patient, at least one structured interactive form, wherein the structured interactive form is related to at least one health related issue of the at least one database. The method also includes receiving via the cloud server, at least one modification to the structured interactive form from the user equipment of the patient, wherein the at least one modification includes a masked identification of the patient.

The method may also transfer the at least one modification at least one of to and from the at least one database, and or determine via the cloud server, a review criticality of the at least one modification to the structured interactive form related to the at least one health related issue. The method may further alert via the cloud server, the user equipment of the patient and a local health care provider server if a review criticality exceeds a predetermined criticality threshold and or display on the user equipment of the patient a follow-on at least one structured interactive form. The at least one structured interactive form may be set by a local health care provider, the issuing of the push notification may be related to a proximate location of the user equipment of the patient to a local health care provider server and the at least one database includes at least one of an unread message database, a reply form database and an encounter summary database.

An example non-transitory computer readable medium comprises instructions that, when read by a processor, cause the processor to perform, issuing a push notification via a cloud server, to a user equipment of a patient and connecting the user equipment of the patient to at least one database that is accessible by the cloud server. The processor also performs displaying on the user equipment of the patient, at least one structured interactive form, wherein the structured interactive form is related to at least one health related issue of the at least one database. The method additionally performs receiving via the cloud server, at least one modification to the structured interactive form from the user equipment of the patient, wherein the at least one modification includes a masked identification of the patient.

The processor may also transfer the at least one modification at least one of to and from the at least one database, and or determine via the cloud server, a review criticality of the at least one modification to the structured interactive form related to the at least one health related issue. The processor may further alert via the cloud server, the user equipment of the patient and a local health care provider server if a review criticality exceeds a predetermined criticality threshold and or display on the user equipment of the patient a follow-on at least one structured interactive form. The at least one structured interactive form may be set by a local health care provider, the issuing of the push notification may be related to a proximate location of the user equipment of the patient to a local health care provider server and the at least one database includes at least one of an unread message database, a reply form database and an encounter summary database.

An example system includes at least one cloud based processor, and at least one memory electrically coupled to the at least one cloud based processor and storing an application, wherein the at least one cloud based processor performs operations to issue a push notification via a cloud server, to a user equipment of a patient and connect the user equipment of the patient to at least one database that is accessible by the cloud server. The processor may also display on the user equipment of the patient, at least one structured interactive form, wherein the structured interactive form is related to at least one health related issue of the at least one database and receive via the cloud server, at least one modification to the structured interactive form from the user equipment of the patient, wherein the at least one modification includes a masked identification of the patient.

The processor may also transfer the at least one modification at least one of to and from the at least one database, and or determine via the cloud server, a review criticality of the at least one modification to the structured interactive form related to the at least one health related issue. The processor may further alert via the cloud server, the user equipment of the patient and a local health care provider server if a review criticality exceeds a predetermined criticality threshold and or display on the user equipment of the patient a follow-on at least one structured interactive form. The at least one structured interactive form may be set by a local health care provider, the issuing of the push notification may be related to a proximate location of the user equipment of the patient to a local health care provider server and the at least one database includes at least one of an unread message database, a reply form database and an encounter summary database.

An example method, comprising requesting via a cloud server, at least one patient response to at least one query of at least one health related issue sent via a direct message channel to a user equipment of a patient, wherein an identity of the patient is masked and wherein a data traffic related to the at least one response and the at least one query accrue at the cloud server. The method includes receiving asynchronously via the cloud server, the at least one patient response to the at least one query from the user equipment in the direct message channel, determining via the cloud server, a review criticality of the at least one patient response related to the at least one health related issue and directly messaging instructions via the cloud server, to the user equipment of the patient if the review criticality exceeds a predetermined criticality threshold, wherein the messaging instructions are related to a proximate location of the user equipment of the patient to a local health care provider server. The method further includes receiving asynchronously via the cloud server, at least one follow-on patient response to the messaging instructions, determining via the cloud server, whether the at least one follow-on patient response reduces the review criticality below the predetermined criticality threshold and alerting via the cloud server, the user equipment of the patient and the local health care provider server if the review criticality of the at least one follow-on patient response continues to exceed the predetermined criticality threshold.

The method may also embed patient specific data via the cloud server, in the directly messaged instructions. The method may alter via the cloud server the direct message channel if the review criticality exceeds an alarm criticality threshold and or alter via the cloud server the direct message channel to the local health care provider server if the review criticality exceeds an alarm criticality threshold. The method may further perform sending via the cloud server a health care provider message based on a health care provider office, sending at least one follow-on query via the cloud server, based on the review criticality and on a health care provider office and escalating via the cloud server the review criticality based on a patient response delay. The method may additionally flag via the cloud server the at least one patient response as at least one critical flagged response if the review criticality exceeds the predetermined criticality threshold for the at least one health related issue.

An example non-transitory computer readable medium comprising instructions that, when read by a processor, cause the processor to perform requesting via a cloud server, at least one patient response to at least one query of at least one health related issue sent via a direct message channel to a user equipment of a patient, wherein an identity of the patient is masked and wherein a data traffic related to the at least one response and the at least one query accrue at the cloud server. The instructions include receiving asynchronously via the cloud server, the at least one patient response to the at least one query from the user equipment in the direct message channel, determining via the cloud server, a review criticality of the at least one patient response related to the at least one health related issue and directly messaging instructions via the cloud server, to the user equipment of the patient if the review criticality exceeds a predetermined criticality threshold, wherein the messaging instructions are related to a proximate location of the user equipment of the patient to a local health care provider server. The instructions further include receiving asynchronously via the cloud server, at least one follow-on patient response to the messaging instructions, determining via the cloud server, whether the at least one follow-on patient response reduces the review criticality below the predetermined criticality threshold and alerting via the cloud server, the user equipment of the patient and the local health care provider server if the review criticality of the at least one follow-on patient response continues to exceed the predetermined criticality threshold.

The non-transitory computer readable medium may further include instructions to embed patient specific data via the cloud server, in the directly messaged instructions and or alter via the cloud server the direct message channel if the review criticality exceeds an alarm criticality threshold. The instructions may also include altering via the cloud server the direct message channel to the local health care provider server if the review criticality exceeds an alarm criticality threshold. The instructions may further include sending via the cloud server a health care provider message based on a health care provider office and sending at least one follow-on query via the cloud server, based on the review criticality and on a health care provider office. The instructions may further include escalating via the cloud server the review criticality based on a patient response delay and or flagging via the cloud server the at least one patient response as at least one critical flagged response if the review criticality exceeds the predetermined criticality threshold for the at least one health related issue.

An example system, comprising at least one cloud based processor, and at least one memory electrically coupled to the processor and storing an application, wherein the processor performs operations to request via a cloud server, at least one patient response to at least one query of at least one health related issue sent via a direct message channel to a user equipment of a patient, wherein an identity of the patient is masked and wherein a data traffic related to the at least one response and the at least one query accrue at the cloud server. The system also receives asynchronously via the cloud server, the at least one patient response to the at least one query from the user equipment in the direct message channel, determines via the cloud server, a review criticality of the at least one patient response related to the at least one health related issue and directly message instructions via the cloud server, to the user equipment of the patient if the review criticality exceeds a predetermined criticality threshold, wherein the message instructions are related to a proximate location of the user equipment of the patient to a local health care provider server. The system instructions further receive asynchronously via the cloud server, at least one follow-on patient response to the message instructions, determine via the cloud server, whether the at least one follow-on patient response reduces the review criticality below the predetermined criticality threshold and alert via the cloud server, the user equipment of the patient and the local health care provider server if the review criticality of the at least one follow-on patient response continues to exceed the predetermined criticality threshold.

The system processor may embed patient specific data via the cloud server, in the directly messaged instructions and may alter via the cloud server the direct message channel if the review criticality exceeds an alarm criticality threshold. The system may flag via the cloud server the at least one patient response as at least one critical flagged response if the review criticality exceeds the predetermined criticality threshold for the at least one health related issue.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

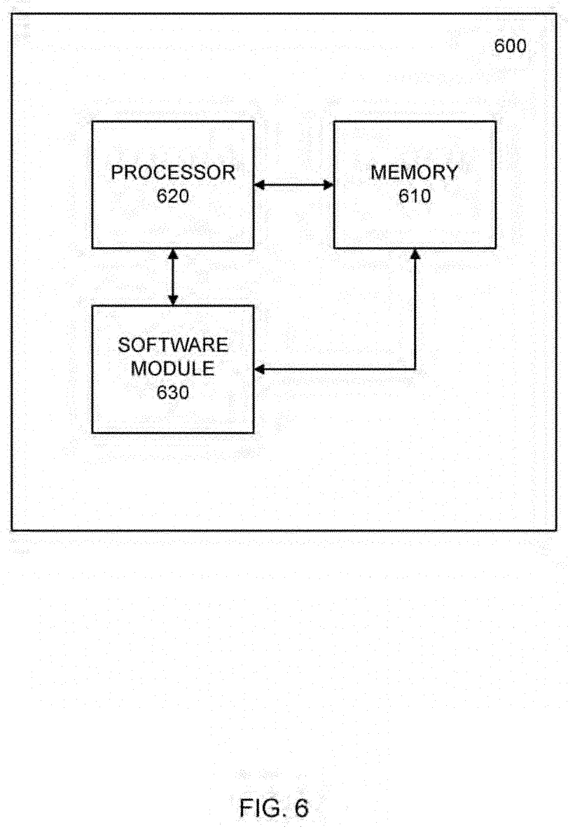

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example network element, which may represent any of the above-described network components of the other figures.

As illustrated in FIG. 6, a memory 610 and a processor 620 may be discrete components of the network entity 600 that are used to execute an application or set of operations. The application may be coded in software in a computer language understood by the processor 620, and stored in a computer readable medium, such as, the memory 610. The computer readable medium may be a non-transitory computer readable medium that includes tangible hardware components in addition to software stored in memory. Furthermore, a software module 630 may be another discrete entity that is part of the network entity 600, and which contains software instructions that may be executed by the processor 620. In addition to the above noted components of the network entity 600, the network entity 600 may also have a transmitter and receiver pair configured to receive and transmit communication signals (not shown).

Although an exemplary embodiment of the system, method, and computer readable medium of the present application has been illustrated in the accompanied drawings and described in the foregoing detailed description, it will be understood that the application is not limited to the embodiments disclosed, but is capable of numerous rearrangements, modifications, and substitutions without departing from the spirit or scope of the application as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way, but is intended to provide one example of many embodiments of the present application. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed, but is merely representative of selected embodiments of the application.

One having ordinary skill in the art will readily understand that the application as discussed above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent, while remaining within the spirit and scope of the application. In order to determine the metes and bounds of the application, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A method, comprising:
requesting via a cloud server at least one patient response to at least one query of at least one health related issue sent to a user equipment of a patient;
receiving via the cloud server at least one patient response to the at least one query from the user equipment;
determining via the cloud server a review criticality of the at least one patient response based on the at least one health related issue;
flagging via the cloud server the at least one patient response as at least one critical flagged response if the review criticality exceeds a predetermined criticality threshold for the at least one health related issue;
disclosing via the cloud server to a local health care provider server the at least one critical flagged response in an order of criticality, thereby increasing workflow efficiency; and outreaching via the cloud server to at least one of the local health care provider server and a local health care provider device the at least one patient response if the review criticality indicates a substantial public hazard.

2. The method of claim 1 wherein the at least one query to the user equipment coincides with a back-to-work schedule of the patient.

3. The method of claim 1 further comprising determining via the cloud server a comprehension of at least one patient response to the at least one query sent from the user equipment.

4. The method of claim 1 further comprising determining via the cloud server a containment compliance of at least one patient response to a set of health care instructions sent to the user equipment.

5. The method of claim 1 further comprising alerting an employer user equipment if the at least one patient response does not indicate a containment compliance of the at least one patient response sent from the user equipment.

6. The method of claim 1 further comprising determining via the cloud server if a cluster of similar at least one critical flagged responses are detected within a workplace of the patient.

7. The method of claim 1 further comprising determining via the cloud server if the patient repeats similar at least one critical flagged responses.

8. The method of claim 1 further comprising determining via the cloud server if a cluster of similar at least one critical flagged responses is detected within a group of at least one of a workplace, a region and a condition.

9. A non-transitory computer readable medium comprising instructions that, when read by a processor, cause the processor to perform:
requesting via a cloud server at least one patient response to at least one query of at least one health related issue sent to a user equipment of a patient;
receiving via the cloud server at least one patient response to the at least one query from the user equipment;
determining via the cloud server a review criticality of the at least one patient response based on the at least one health related issue;
flagging via the cloud server the at least one patient response as at least one critical flagged response if the review criticality exceeds a predetermined criticality threshold for the at least one health related issue;
disclosing via the cloud server to a local health care provider server the at least one critical flagged response in an order of criticality; and
outreaching via the cloud server to at least one of the local health care provider server and a local health care provider device the at least one patient response if the review criticality indicates a substantial public hazard.

10. The non-transitory computer readable medium of claim 9, further comprising determining via the cloud server a comprehension of the at least one patient response to the at least one query sent from the user equipment.

11. The non-transitory computer readable medium of claim 9, further comprising determining via the cloud server a comprehension of the at least one patient response to the at least one query sent from the user equipment.

12. The non-transitory computer readable medium of claim 9, further comprising determining via the cloud server a containment compliance of the at least one patient response to a set of health care instructions sent to the user equipment.

13. The non-transitory computer readable medium of claim 9, further comprising alerting an employer user equipment if the at least one patient response does not indicate a containment compliance of the at least one patient response sent from the user equipment.

14. The non-transitory computer readable medium of claim 9, further comprising determining via the cloud server if a cluster of similar at least one critical flagged responses are detected within a workplace of the patient.

15. The non-transitory computer readable medium of claim 9, further comprising determining via the cloud server if the patient repeats similar at least one critical flagged responses.

16. The non-transitory computer readable medium of claim 9, further comprising determining via the cloud server if a cluster of similar at least one critical flagged responses is detected within a group of at least one of a workplace, a region and a condition.

17. A system, comprising:
at least one cloud based processor; and
at least one memory electrically coupled to the at least one processor to store an application,
wherein the processor performs operations to:
request via a cloud server at least one patient response to at least one query of at least one health related issue sent to a user equipment of a patient;
receive via the cloud server at least one patient response to the at least one query from the user equipment;
determine via the cloud server a review criticality of the at least one patient response based on the at least one health related issue;
flag via the cloud server the at least one patient response as at least one critical flagged response if the review criticality exceeds a predetermined criticality threshold for the at least one health related issue;
disclose via the cloud server to a local health care provider server the at least one critical flagged response in an order of criticality; and
outreach via the cloud server to at least one of the local health care provider server and a local health care provider device the at least one patient response if the review criticality indicates a substantial public hazard.

18. The system of claim 17, wherein the processor further performs an operation to determine via the cloud server a comprehension of the at least one patient response of the workplace to the at least one query sent from the user equipment.

19. The system of claim 17, wherein the processor further performs an operation to at least one of determine via the cloud server a comprehension of the at least one patient response of the workplace to the at least one query sent from the user equipment and determine via the cloud server a containment compliance of the at least one patient response of the workplace to a set of health care instructions sent to the user equipment.

20. The system of claim 17, wherein the processor further performs an operation to determine via the cloud server if a cluster of similar at least one critical flagged responses are detected within the workplace of the patient.
